(19) United States
(12) Patent Application Publication
(10) Pub. No.: US 2025/0281082 A1
(43) Pub. Date: Sep. 11, 2025

(54) ENDOSCOPE SYSTEM, METHOD OF GENERATING BIOLOGICAL PARAMETER IMAGE, AND NON-TRANSITORY COMPUTER READABLE MEDIUM

(71) Applicant: FUJIFILM Corporation, Tokyo (JP)

(72) Inventor: Takaaki SAITO, Kanagawa (JP)

(73) Assignee: FUJIFILM Corporation, Tokyo (JP)

(21) Appl. No.: 19/070,441

(22) Filed: Mar. 4, 2025

(30) Foreign Application Priority Data
Mar. 5, 2024 (JP) 2024-033324

(51) Int. Cl. A61B 5/1455; A61B 1/00; A61B 1/06

(52) U.S. Cl. CPC A61B 5/14552; A61B 1/000094; A61B 1/0638

(57) ABSTRACT

An endoscope system includes a processor, in which the processor generates an aligned first image by performing a registration process on a first endoscope image based on a plurality of second endoscope images, acquires an aligned first image set including a plurality of the aligned first images, calculates oxygen saturation of an observation target based on the acquired aligned monochromic image set, generates a biological parameter image based on the oxygen saturation, and performs control of displaying the biological parameter image on a display.

### CROSS-REFERENCE TO RELATED APPLICATIONS

This application claims priority under 35 U.S.C § 119 (a) to Japanese Patent Application No. 2024-033324 filed on 5 Mar. 2024. The above application is hereby expressly incorporated by reference, in its entirety, into the present application.

### BACKGROUND OF THE INVENTION

### 1. Field of the Invention

The disclosure relates to an endoscope system, a method of generating a biological parameter image, and a non-transitory computer readable medium.

### 2. Description of the Related Art

In recent years, in a medical field using an endoscope, a technique of generating a biological parameter image, such as oxygen saturation imaging, has become known. The oxygen saturation imaging is a technique of calculating hemoglobin oxygen saturation from a small amount of spectral information, such as two or three, of visible light.

As the oxygen saturation imaging, a technique of generating an oxygen saturation image in which an influence of a yellow coloring agent that causes a problem during endoscopic observation is corrected by using image signals of four wavelength ranges in order to calculate accurate oxygen saturation is known (JP2015-177961A, corresponding to US2015/0238126A1). In addition, a technique of calculating a movement amount of an observation target using a first image signal and a second image signal obtained by imaging the observation target while being illuminated with two illumination light beams, that is, first illumination light and second illumination light, and then calculating oxygen saturation using a method according to the movement amount is known (JP2016-185398A).

### SUMMARY OF THE INVENTION

With the advancement of image processing technique, there has been a demand for generating a biological parameter image with higher accuracy and higher robustness.

An object of the disclosure is to provide an endoscope system, a method of generating a biological parameter image, and a non-transitory computer readable medium capable of generating a biological parameter image with higher accuracy and higher robustness.

According to an exemplary embodiment of the invention, there is provided an endoscope system comprising: a light source device that alternately emits one of a plurality of first illumination light beams and a second illumination light beam having a wider band than the first illumination light beams as illumination light; an endoscope including an imaging sensor that images an observation target illuminated with the illumination light; a processor device including a processor that generates a biological parameter image based on an endoscope image obtained by the imaging sensor and that performs control of displaying the biological parameter image on a display; and the display, in which the plurality of first illumination light beams are light beams in wavelength ranges having different dependences on oxygen saturation, and the processor generates an aligned first image by performing a registration process on a first endoscope image obtained by imaging the observation target illuminated with the first illumination light beam, based on a plurality of second endoscope images obtained by imaging the observation target illuminated with the second illumination light beam, acquires an aligned first image set including a plurality of the aligned first images, calculates oxygen saturation of the observation target based on the acquired aligned first image set, and generates the biological parameter image based on the oxygen saturation.

It is preferable that the registration process includes a movement amount calculation process and a movement amount correction process, the movement amount calculation process is a process of calculating a movement amount of the first endoscope image based on the plurality of second endoscope images, and the movement amount correction process is a process of generating the aligned first image by performing correction based on the movement amount on the first endoscope image of which the movement amount is calculated.

It is preferable that the light source device alternately emits the second illumination light beam and any of the plurality of first illumination light beams for each frame. It is preferable that a central wavelength of each of the plurality of first illumination light beams is any one of 450 nm, 470 nm, 540 nm, 620 nm, 690 nm, or 850 nm. It is preferable that there are five first illumination light beams, and central wavelengths of the first light beams are 470 nm, 540 nm, 620 nm, 690 nm, and 850 nm.

It is preferable that the light source device illuminates the observation target with light-emitting units that emit all of the plurality of first illumination light beams in a preset order, and the processor generates the aligned first image set based on a plurality of the first endoscope images obtained in the light-emitting units. It is preferable that the light source device illuminates the observation target with the light-emitting units that emit all of the plurality of first illumination light beams in ascending order of a central wavelength. It is preferable that the second illumination light beam is generated by emitting two or more first illumination light beams among the plurality of first illumination light beams. It is preferable that the second illumination light beam is generated by simultaneously emitting first illumination light beams having central wavelengths of 450 nm, 540 nm, and 620 nm among the plurality of first illumination light beams.

It is preferable that the movement amount calculation process is a process of calculating the movement amount of the first endoscope image based on two second endoscope images obtained in frames immediately before and after the first endoscope image. It is preferable that the processor calculates the oxygen saturation of the observation target for each pixel of the aligned first image included in the aligned first image set.

It is preferable that the endoscope system has a normal observation mode in which the light source device continuously emits the second illumination light beam and the processor performs control of continuously displaying the second endoscope image on the display, and a biological parameter image acquisition mode in which the light source device alternately emits the second illumination light and any of the plurality of first illumination light beams and the processor performs control of displaying the biological parameter image on the display, and the endoscope includes a switching operation unit for switching between the normal observation mode and the biological parameter image acquisition mode, the switching operation unit being operated by an endoscope operator.

It is preferable that, in a case in which the endoscope operator switches a mode from the normal observation mode to the biological parameter image acquisition mode by operating the switching operation unit, the processor generates the biological parameter image based on the aligned first image set acquired first after the switching, performs control of displaying the generated biological parameter image on the display, and then automatically returns to the normal observation mode.

It is preferable that the light source device illuminates the observation target with light-emitting units that emit all of the plurality of first illumination light beams in a preset order, and the processor calculates a total movement amount obtained by adding up movement amounts in a plurality of the first endoscope images obtained in the light-emitting units, acquires the aligned first image set including the plurality of aligned first images obtained in the light-emitting units in a case in which the total movement amount is within a preset range, and calculates the oxygen saturation based on the acquired aligned first image set.

It is preferable that the light source device illuminates the observation target with light-emitting units that emit all of the plurality of first illumination light beams in a preset order, and the processor calculates a total movement amount obtained by adding up movement amounts in a plurality of the first endoscope images obtained in the light-emitting units, and performs adjustment to reduce a resolution of the aligned first image based on which the oxygen saturation of the observation target is calculated such that a degree of reduction in the resolution increases as the total movement amount increases.

According to the exemplary embodiment of the invention, there is provided a method of generating a biological parameter image executed in an endoscope system including a light source device, an endoscope, and a processor device, in which the light source device alternately emits one of a plurality of first illumination light beams and a second illumination light beam having a wider band than the first illumination light beams as illumination light, the endoscope includes an imaging sensor that images an observation target illuminated by the light source device, the processor device includes a processor that generates a biological parameter image based on an endoscope image obtained by the imaging sensor, and the plurality of first illumination light beams are light beams in wavelength ranges having different dependences on oxygen saturation, the method comprising: via the processor, a step of generating an aligned first image by performing a registration process on a first endoscope image obtained by imaging the observation target illuminated with the first illumination light beam, based on a plurality of second endoscope images obtained by imaging the observation target illuminated with the second illumination light beam; a step of acquiring an aligned first image set including a plurality of the aligned first images; a step of calculating oxygen saturation of the observation target based on the aligned first image set; and a step of generating the biological parameter image based on the oxygen saturation.

According to the exemplary embodiment of the invention, there is provided a non-transitory computer readable medium for storing a computer-executable program for an endoscope system including a light source device that alternately emits one of a plurality of first illumination light beams and a second illumination light beam having a wider band than the first illumination light beams as illumination light, an endoscope including an imaging sensor that images an observation target illuminated with the illumination light, and a processor device including a processor that generates a biological parameter image based on an endoscope image obtained by the imaging sensor and that performs control of displaying the biological parameter image on a display, the computer-executable program being executed by the processor and causing the processor to execute a function of: generating an aligned first image by performing a registration process on a first endoscope image obtained by imaging the observation target illuminated with the first illumination light beam, based on a plurality of second endoscope images obtained by imaging the observation target illuminated with the second illumination light beam; acquiring an aligned first image set including a plurality of the aligned first images; calculating oxygen saturation of the observation target based on the aligned first image set; generating the biological parameter image based on the oxygen saturation; and performing control of displaying the biological parameter image on the display.

According to the exemplary embodiments of the invention, it is possible to generate a biological parameter image with higher accuracy and higher robustness.

### BRIEF DESCRIPTION OF THE DRAWINGS

FIG. 1 is an explanatory diagram illustrating registration of monochromic images.

FIG. 2 is a schematic diagram of an endoscope system for a digestive tract.

FIG. 3 is an explanatory diagram illustrating a display aspect of an endoscope image on a display and an extended display in a normal observation mode.

FIG. 4 is an explanatory diagram showing a display aspect of an endoscope image on a display and an extended display in an oxygen saturation mode.

FIG. 5 is an image diagram of an extended display that displays an internal digestive tract oxygen saturation image on a left side and a serous membrane-side oxygen saturation image on a right side.

FIG. 6 is a block diagram showing functions of the endoscope system.

FIG. 7 is a graph showing light emission spectra of monochromic light beams b, sb, g, a, r, and ir.

FIG. 8 is an explanatory diagram showing a light emission pattern in the oxygen saturation mode.

FIG. 9 is a graph showing spectral sensitivity of an imaging sensor.

FIG. 10 is a table showing image signals obtained in the normal observation mode.

FIG. 11 is a table showing spectral image signals obtained in the oxygen saturation mode.

FIG. 12 is a graph showing a relationship between central wavelengths of monochromic light beams b, sb, g, a, r, and ir and a reflectivity of hemoglobin.

FIG. 13 is a block diagram showing functions of an extended processor device.

FIG. 14 is an explanatory diagram illustrating calculation of a movement amount.

FIG. 15 is an explanatory diagram illustrating movement amount correction based on a change in illuminance.

FIG. 16 is a graph showing a relationship between a reflectivity R and a ratio μa/μs′.

FIG. 17 is a flowchart showing a series of flows in the oxygen saturation mode.

FIG. 18 is an explanatory diagram showing an oxygen saturation, a hemoglobin concentration, a bilirubin concentration, and a scattering wavelength-dependent parameter of a spectral model minimally required for calculating a biological parameter.

FIG. 19 is an explanatory diagram showing an oxygen saturation, a hemoglobin concentration, a bilirubin concentration, and a scattering wavelength-dependent parameter of a spectral model required for improving calculation accuracy of a biological parameter.

FIG. 20 is an explanatory diagram showing an oxygen saturation, a hemoglobin concentration, a bilirubin concentration, and a scattering wavelength-dependent parameter of a spectral model required for further improvement of accuracy in calculation of a biological parameter.

FIG. 21 is a graph showing a spectral reflectivity determined by a spectral model.

FIG. 22 is a block diagram showing functions of an extended processor device according to a second embodiment.

FIG. 23 is a table showing a standard spectral signal for each spectral model.

FIG. 24 is a graph showing a spectral reflectivity determined by a phantom.

FIG. 25 is a block diagram showing functions of an extended processor device according to a third embodiment.

FIG. 26 is a table showing a standard spectral signal for each phantom.

FIG. 27 is a block diagram showing functions of an extended processor device according to a fourth embodiment.

FIG. 28 is an explanatory diagram showing a method of acquiring a standard spectral signal in the fourth embodiment.

FIG. 29 is a schematic diagram of an endoscope system for laparoscopy.

FIG. 30 is an explanatory diagram showing an imaging unit including three monochromic imaging sensors.

FIG. 31 is an explanatory diagram showing an imaging unit including one color imaging sensor.

FIG. 32 is a graph showing a light emission spectrum of illumination light having a wide band.

FIG. 33 is an explanatory diagram showing a pixel array in a spectroscopic imaging sensor.

FIG. 34 is an explanatory diagram showing another pattern of a light emission pattern in the oxygen saturation mode.

### DESCRIPTION OF THE PREFERRED EMBODIMENTS

### First Embodiment

An example of an endoscope system according to an embodiment of the invention will be described. First, the background leading to obtaining one aspect of the following embodiment will be described. Oxygen saturation imaging using an endoscope is a technique of calculating hemoglobin oxygen saturation from a small amount of spectral information of visible light. In the related art, a technique of creating and displaying an oxygen saturation image from two or three spectral signals acquired while switching illumination light in two or three frames of a video of an endoscope image acquired by an endoscope is known. However, in a case in which an attempt is made to create an oxygen saturation image with higher accuracy and higher robustness using more spectral signals, in order to acquire many spectral images, it is necessary to acquire the images by switching illumination light in more frames. In such a case, it is considered that an influence of the misregistration between the images corresponding to different wavelengths becomes greater in a spectral image set for creating a biological parameter image such as the oxygen saturation image.

That is, by acquiring many spectral images and acquiring a biological parameter such as oxygen saturation based on the spectral images, the biological parameter can be calculated with high accuracy, but there is a concern that the influence of the misregistration between the spectral images becomes greater.

An endoscope system, a method of generating a biological parameter image, and a program according to an embodiment of the invention are each configured to comprise a specific light source device, an endoscope, a processor device, and a display, so that conflicting characteristics such as high accuracy in biological parameters such as oxygen saturation and a hemoglobin concentration and high robustness against movement of an observation target and/or the endoscope can be achieved, and an oxygen saturation image or the like can be generated and displayed on the display with higher accuracy and high robustness.

The endoscope system according to the embodiment of the invention comprises a light source device, an endoscope, a processor device, and a display. These devices are connected to each other and can communicate with each other as necessary. The connection or communication may be wired or wireless. In addition, the endoscope system may be any type of endoscope system as long as the endoscope system acquires a biological parameter image such as an oxygen saturation image. Therefore, the endoscope system may be any of an upper endoscope, a lower endoscope, or a laparoscope.

In the present specification, the biological parameter image refers to an image obtained by visualizing biological parameters such as oxygen saturation and hemoglobin concentration of an observation target using a heat map, numerical display, or the like. Therefore, the oxygen saturation image refers to an image obtained by visualizing the oxygen saturation of the observation target using a heat map or the like. In addition, the endoscope image refers to an image obtained by an endoscope, and includes a still image and a video.

The light source device alternately emits a plurality of first illumination light beams and any of second illumination light beams having a wider band than the first illumination light beams as illumination light. In the present embodiment, a monochromic light beam is emitted as the first illumination light beam, and a polychromic light beam is emitted as the second illumination light beam. Therefore, the light source device continuously emits the illumination light in the order of a polychromic light beam, any monochromic light beam, a polychromic light beam, and any monochromic light beam. The polychromic light beam is normal light that is normally used in a case in which an endoscope operator(hereinafter, referred to as a user) performs observation with the endoscope, and is illumination light with which the observation target can be observed in natural colors. Therefore, the polychromic light beam is preferably white light obtained by emitting a plurality of monochromic light beams. In the present specification, the white light also includes white-equivalent light having fewer short wavelength components than the white light that is normally used. The white-equivalent light is also normal light with which the observation target can be observed in natural colors.

The plurality of monochromic light beams are a plurality of light beams having different wavelength ranges. In addition, the plurality of monochromic light beams are light beams in wavelength ranges having different dependences on the oxygen saturation. One piece of spectral information can be obtained from one monochromic light beam. The plurality of monochromic light beams consist of two or more monochromic light beams. Using a larger number of monochromic light beams is preferable because it results in more image information being obtained and the calculated biological parameter will be more accurate, but it is preferable that the number of the monochromic light beams is so large that the effects of the invention are not impaired due to the complexity of the light source device and the calculation of the biological parameter. For example, in order to preferably calculate the biological parameter such as oxygen saturation, the plurality of monochromic light beams are preferably three or more types having different wavelength ranges.

The endoscope includes an imaging sensor that images the observation target illuminated with the illumination light emitted by the light source device. A unit in which the imaging sensor generates a still image, which is one endoscope image, in one exposure period is defined as one frame. Therefore, the frames are obtained in time series. In the endoscope system, the light source device, the imaging sensor, and the like are synchronized, so that an endoscope image of the observation target illuminated with each illumination light can be obtained. For example, still images are successively generated, one frame at a time, in the order of a still image illuminated with a polychromic light beam, a still image illuminated with any monochromic light beam, a still image illuminated with a polychromic light beam, and a still image illuminated with any monochromic light beam.

The processor device includes a processor. The processor generates a biological parameter image by performing processing based on an endoscope image obtained by the imaging sensor and control of displaying the biological parameter image on the display. The biological parameter image is generated based on an endoscope image which is a still image acquired by the endoscope.

The processor first generates an aligned monochromic image (aligned first image) by performing a registration process on a monochromic endoscope image (first endoscope image) (hereinafter, referred to as a monochromic image) based on a plurality of multicolor endoscope images (second endoscope images) (hereinafter, referred to as polychromic images) obtained by imaging the observation target illuminated with a polychromic light beam. The monochromic image is an endoscope image obtained by imaging the observation target illuminated with a monochromic light beam. Therefore, the aligned monochromic image is a spectral image and is an image obtained by performing the registration process on the monochromic image. A plurality of the aligned monochromic images are obtained, and these are set as an aligned monochromic image set (aligned first image set), and the oxygen saturation of the observation target is calculated based on the aligned monochromic image set.

In order to perform the registration, information on a movement amount between two images to be registered is required. However, since wavelengths of the illumination light differ between the monochromic images to be registered, an appearance of contrast of a blood vessel of an observation target tissue in the image is completely different depending on the image. For example, in a case of blue light, green light, and the like included in white light which is illumination light, the blood vessel of the observation target is clearly shown in the obtained endoscope image, but, in a case of red light, near-infrared light, and the like, the blood vessel is not clearly shown. Therefore, there is an image in which it is difficult to extract feature points for calculating the movement amount between the images. Therefore, the movement amount between frame images captured with a monochromic light beam is calculated using a frame image obtained using a polychromic light beam which is common illumination light.

Since the polychromic image is an image obtained by imaging the observation target illuminated with the same illumination light such as white light, it can be said that the object shown in the image is more uniform and the image information is relatively large than in a case of being illuminated with a monochromic light beam. As described above, since the plurality of monochromic light beams are light beams having different wavelength ranges, in a case in which each of the monochromic light beams is used as illumination light, a different object may be shown in the obtained image.

Therefore, it is possible to more accurately grasp misregistration between images by comparing polychromic images rather than comparing monochromic light beams with which different objects are shown. As described above, by performing the registration process on the monochromic image using a plurality of polychromic images as a reference, it is possible to perform the registration process with high accuracy even in a case in which there are a plurality of monochromic images. In an aligned monochromic image, which is a monochromic image after the registration process, the misregistration based on the movement of the observation target, the movement of the endoscope, and the like is processed and eliminated.

As a method of performing the registration process on the monochromic image based on the polychromic images, various types of processing can be employed as long as the registration process can be performed on the monochromic image based on the polychromic images. For example, the registration process can be performed by using any selected monochromic image among a plurality of the monochromic images as a reference, and performing the registration so that several frames of monochromic images before and after the reference monochromic image match the selected monochromic image.

As an example of the registration process, first, a monochromic image is set as an even-numbered frame, a movement amount between odd-numbered frames of a polychromic image adjacent to the monochromic image is calculated, and, based on the movement amount, a position of the even-numbered frame of the monochromic image interposed between the odd-numbered frames is estimated. Subsequently, a movement amount between the monochromic images is calculated based on the estimated positions of the even-numbered frames of the plurality of monochromic images. Then, one frame of the plurality of monochromic images can be used as a reference, and the other monochromic images can be registered based on the movement amount with respect to the frame used as the reference.

In this case, it is preferable that the registration process includes a movement amount calculation process and a movement amount correction process. In the movement amount calculation process, for example, the movement amount calculation process is performed between two frames of the polychromic images captured before and after the monochromic image is acquired, that is, in time series, before and after a time point at which the monochromic image is captured, and a movement amount between the polychromic images is obtained.

Specifically, it is preferable that the movement amount calculation process is performed by dividing the polychromic image into a plurality of predetermined regions. A method of calculating the movement amount will be described below. Then, the movement amount of the monochromic image is estimated using the obtained movement amount. That is, based on two frames of the polychromic images having different imaging times, the movement amount can be estimated by interpolation for the monochromic image captured between time points at which the polychromic images are captured. As a method of the interpolation, a generally used method such as linear interpolation or various types of complementation is appropriately used.

The movement amount correction process is a process of correcting the movement amount of each of the plurality of monochromic images by using the monochromic images whose movement amount is estimated. For example, one reference monochromic image (hereinafter, referred to as a reference monochromic image) is selected from among the plurality of monochromic images whose movement amount is estimated, and the other monochromic images can be registered with the reference monochromic image based on each movement amount. The monochromic image that has been subjected to the above-described registration process is an aligned monochromic image.

A plurality of the aligned monochromic images are generated to form an aligned monochromic image set. The aligned monochromic image is a spectral image consisting of a plurality of monochromic light beams, which are light beams in wavelength ranges having different dependencies on the oxygen saturation. Therefore, with the aligned monochromic image set, the aligned monochromic image set includes a plurality of pieces of spectral image information, so that it is possible to calculate a biological parameter such as oxygen saturation with higher accuracy. In addition, since these spectral images are registered with high accuracy, it is possible to calculate a biological parameter with high robustness against the movement of the observation target, the movement of the endoscope, and the like.

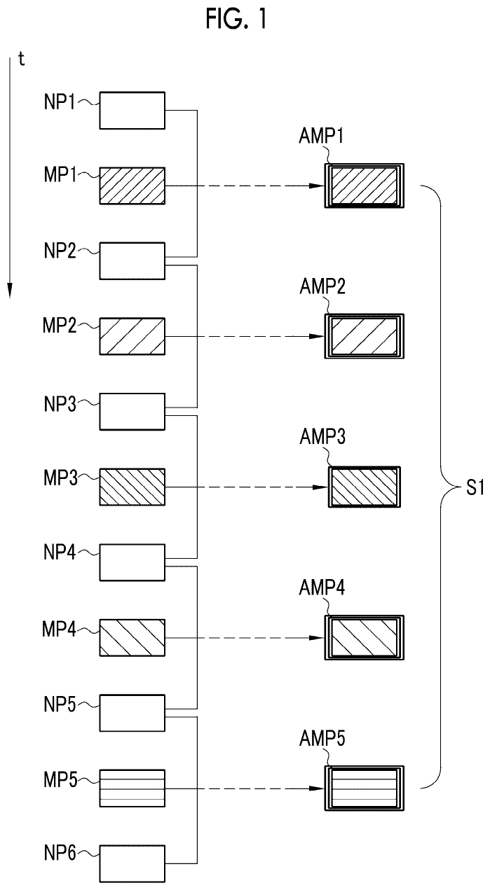

As shown in FIG. 1, specifically, in a case in which the number of a plurality of monochromic light beams is five and an image is captured by switching the illumination light one frame at a time, in a case in which time t at which the image is acquired is indicated in time series, 11 endoscope images are obtained in the following order: a first white light image NP1, which is a polychromic image, a first monochromic image MP1 obtained using a first monochromic light beam, a second white light image NP2, a second monochromic image MP2 obtained using a second monochromic light beam, a third white light image NP3, a third monochromic image MP3 obtained using a third monochromic light beam, a fourth white light image NP4, a fourth monochromic image MP4 obtained using a fourth monochromic light beam, a fifth white light image NP5, a fifth monochromic image MP5 obtained using a fifth monochromic light beam, and a sixth white light image NP6, and thereafter the order in which the monochromic image is obtained using the first monochromic light beam is repeated. In the drawing, different shading indicates endoscope images acquired by illumination light in different wavelength ranges. In addition, in the following description, in a case in which the order of the frames is not specified, the first to fifth white light images are simply referred to as a white light image NP. In addition, the sixth white light image NP6 is the same as the first white light image NP1, and the first monochromic image MP1 is obtained after the sixth white light image NP6 is acquired.

For five monochromic images, that is, the first monochromic image MP1, the second monochromic image MP2, the third monochromic image MP3, the fourth monochromic image MP4, and the fifth monochromic image MP5, a registration process unit 61 (see FIG. 13) performs the registration process by using polychromic images of frames before and after each of the five monochromic images. The registration process unit 61 includes a movement amount calculation unit 64 and a movement amount correction unit 65 (see FIG. 13).

The movement amount calculation unit 64 estimates the movement amount of the monochromic image using the polychromic images of the frames before and after each of the five monochromic images. In the first monochromic image MP1, the movement amount of the first monochromic image MP1 is estimated by interpolation based on the movement amount calculated from the first polychromic image NP1 and the second polychromic image NP2. The first monochromic image MP1 is registered based on the movement amount of the first monochromic image MP1. In a similar manner, the second to fifth monochromic images MP2 to MP5 are registered. A method of the registration will be described below.

The first monochromic image MP1 that has been subjected to the registration is an aligned first monochromic image AMP1. Similarly, an aligned second monochromic image AMP2, an aligned third monochromic image AMP3, an aligned fourth monochromic image AMP4, and an aligned fifth monochromic image AMP5 are generated.

These aligned monochromic images are combined to form one aligned monochromic image set. Oxygen saturation, which is a biological parameter of the observation target, is calculated based on one aligned monochromic image set formed of a plurality of aligned monochromic images including image information based on light beams in wavelength ranges having different dependencies on the oxygen saturation. Therefore, as described above, the biological parameter can be calculated with higher accuracy. Each of the plurality of aligned monochromic images is an image that has been subjected to the registration based on the polychromic images. Therefore, the biological parameter can be calculated with higher accuracy and higher robustness.

The display displays the biological parameter image with almost real-time contents. In addition, by displaying the polychromic image, the display also displays the endoscope image obtained using the polychromic light beam with almost real-time contents. Therefore, in operating the endoscope, the user can perform an operation, a diagnoses, and the like using the endoscope while viewing a natural color endoscope image by the polychromic image displayed on the display and a biological parameter image such as an oxygen saturation image in a manner in which there is no discomfort in almost real-time.

With the above-described configuration, according to the endoscope system and the like according to the embodiment of the invention, it is possible to generate a biological parameter image with higher accuracy and higher robustness.

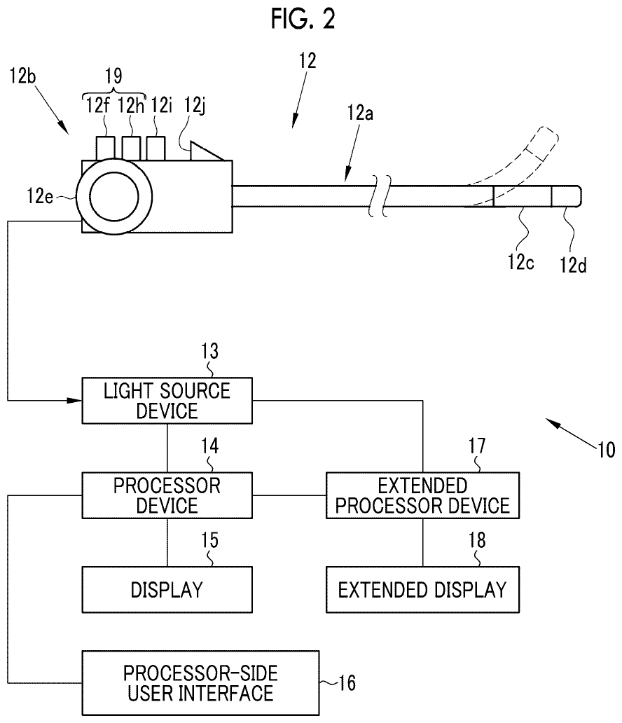

Hereinafter, an embodiment will be described with reference to the drawings. As shown in FIG. 2, an endoscope system 10 comprises an endoscope 12, a light source device 13, a processor device 14, a display 15, a processor-side user interface 16, an extended processor device 17, and an extended display 18. The endoscope 12 is optically or electrically connected to the light source device 13 and electrically connected to the processor device 14. The extended processor device 17 is electrically connected to the light source device 13 and the processor device 14. The display according to the invention includes the extended display 18 in addition to the display 15.

The endoscope 12 has an insertion part 12a, an operating part 12b, a bendable part 12c, and a distal end part 12d. The insertion part 12a is inserted into a body of a subject. The operating part 12b is provided at a base end portion of the insertion part 12a. The bendable part 12c and the distal end part 12d are provided on a distal end side of the insertion part 12a. The bendable part 12c performs a bending operation by operating an angle knob 12e of the operating part 12b. The distal end part 12d is directed in a desired direction of the user by the bending operation of the bendable part 12c. A forceps channel (not shown) for inserting a treatment tool or the like is provided from the insertion part 12a to the distal end part 12d. The treatment tool is inserted into the forceps channel through a forceps port 12j.

An optical system for forming a subject image and an optical system for irradiating the subject with illumination light are provided inside the endoscope 12. The operating part 12b is provided with the angle knob 12e, a mode selector switch 12f, a still image acquisition instruction switch 12h, and a zoom operating part 12i. The mode selector switch 12f is a switching operation unit used for a switching operation of an observation mode.

The endoscope system 10 has a normal observation mode and an oxygen saturation mode which is a biological parameter image acquisition mode, as observation modes, and the switching between the modes is performed by the user operating the mode selector switch 12f. In the normal observation mode, the light source device 13 continuously emits white light, which is a polychromic light beam, and the processor performs control of continuously displaying a polychromic image on the display. In the oxygen saturation mode, the light source device 13 alternately emits white light and any of a plurality of monochromic light beams, and the processor performs control of displaying a biological parameter image on the display. The user can switch between the normal observation mode and the oxygen saturation mode by operating the mode selector switch 12f.

The still image acquisition instruction switch 12h is used for an instruction to acquire a still image of the subject. The zoom operating part 12i is used for an operation of enlarging or reducing an observation target. The mode selector switch 12f and the still image acquisition instruction switch 12h are included in a scope-side user interface 19 for performing various operations on the processor device 14.

The light source device 13 emits illumination light. The processor device 14 performs system control on the endoscope system 10 and further performs image processing or the like on an image signal transmitted from the endoscope 12 to generate an endoscope image or the like. The display 15 displays the endoscope image or the like transmitted from the processor device 14. The processor-side user interface 16 includes a keyboard, a mouse, a microphone, a tablet, a foot switch, a touch pen, and the like, and receives an input operation such as a function setting.

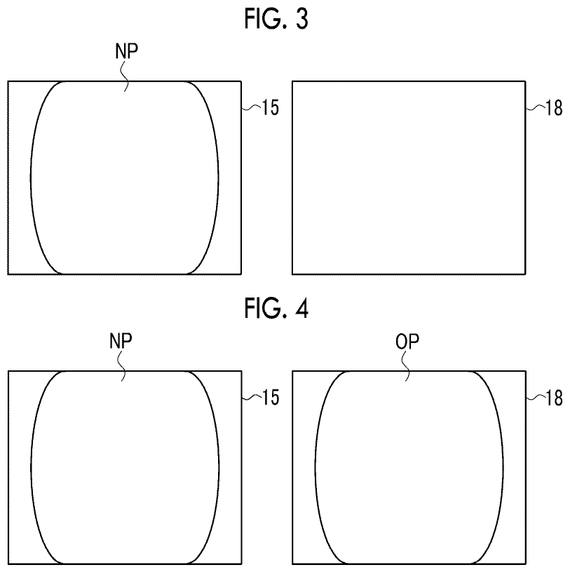

As shown in FIG. 3, in the normal observation mode, a natural color white light image obtained by imaging an observation target using white light as illumination light is displayed on the display 15, while nothing is displayed on the extended display 18. In the drawing, a part of the display 15 or 18 may be shown.

As shown in FIG. 4, in the oxygen saturation mode, oxygen saturation of an observation target is calculated, and an oxygen saturation image OP obtained by visualizing the calculated oxygen saturation is displayed on the extended display 18. In addition, in the oxygen saturation mode, the white light image NP is displayed on the display 15. The oxygen saturation image may be displayed on the display 15.

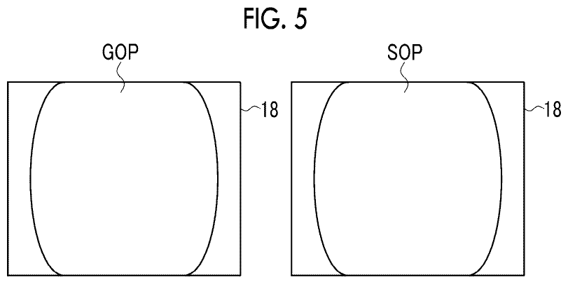

The endoscope system 10 is used for an upper endoscope for the inside of a digestive tract such as a stomach or a large intestine, and the endoscope 12 is a flexible endoscope type. For example, in the oxygen saturation mode, as shown in a left side of FIG. 5, an internal digestive tract oxygen saturation image GOP obtained by imaging a state of the oxygen saturation inside the digestive tract is displayed on the extended display 18. An endoscope system 100 described below is used for laparoscopic surgery or the like for a serous membrane or the like outside an organ, and the endoscope 12, which is a laparoscope, is a rigid endoscope type. For example, in the oxygen saturation mode, as shown in a right side of FIG. 5, a serous membrane-side oxygen saturation image SOP obtained by visualizing a state of the oxygen saturation on the serous membrane side of the large intestine is displayed on the extended display 18.

In the oxygen saturation mode, it is possible to calculate the oxygen saturation more accurately in the following cases.
Case in which a predetermined target part (for example, esophagus, stomach, large intestine) is observed
Case of environment other than extracorporeal environment with illumination therearound
Case in which residues, residual liquids, mucus, blood, and fat do not remain on a mucous membrane and a serous membrane
Case in which no coloring agent is sprayed onto a mucous membrane
Case in which the endoscope 12 is separated from an observation target by a distance of more than 7 mm
Case in which the endoscope 12 observes an observation target at an appropriate distance without being significantly separated from the observation target
Case of a region where illumination light sufficiently reaches an observation target
Case in which specularly reflected light from an observation target is small
Case of a 2/3 internal region of the entire oxygen saturation image
Case in which the movement of the endoscope 12 is small or the movement of a patient or an observation target, such as a heartbeat or breathing, is small
Case in which a blood vessel in a deep portion of a digestive tract mucous membrane is not observed

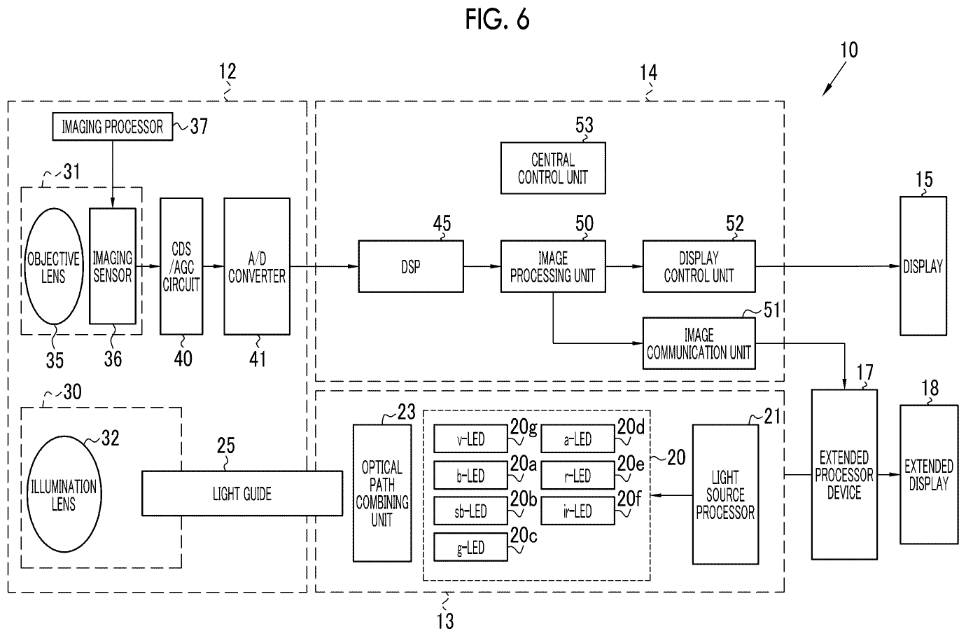

As shown in FIG. 6, the light source device 13 comprises a light source unit 20 and a light source processor 21 that directly controls the light source unit 20. A central control unit 53 of the processor device 14 controls the entire endoscope system 10 and controls the light source processor 21 and the like described below. The extended processor device 17 comprises an imaging processor 37, performs processing based on the endoscope image sent from the processor device 14, and generates a biological parameter image. As a result, components of the endoscope system 10 operate in conjunction with each other.

The light source unit 20 emits at least any of a polychromic light beam or a monochromic light beam as illumination light. The light source unit 20 includes, for example, a plurality of semiconductor light sources, turns on or off each of these semiconductor light sources, and emits illumination light, with which an observation target is illuminated, by controlling the amount of light emitted from each semiconductor light source, in a case of turning on each semiconductor light source. The light source unit 20 comprises a light emitting diode (LED) as a light source, and comprises six LEDs, that is, a violet-light emitting diode (v-LED) 20g, a blue-light emitting diode (b-LED) 20a, a sky blue-light emitting diode (sb-LED) 20b, a green-light emitting diode (g-LED) 20c, an amber-light emitting diode (a-LED) 20d, a red-light emitting diode (r-LED) 20e, and an infra red-light emitting diode (ir-LED) 20f.

The number of the light sources included in the light source unit 20 is not limited to six. Any number may be used as long as a spectral image from which the biological parameter of the oxygen saturation or the hemoglobin concentration can be acquired with high accuracy is obtained, and at least three types of the sb-LED 20b, the g-LED 20c, and the a-LED 20d may be used. In addition to the above-described LEDs, the violet-light emitting diode (v-LED) or the like may be provided for use in illumination light as white light using a polychromic light beam.

Light emitted from each of the LEDs 20a to 20f is incident into a light guide 25 via an optical path combining unit 23 composed of a mirror, a lens, and the like. The light guide 25 is built in the endoscope 12 and a universal cord (a cord connecting the endoscope 12 to the light source device 13 and the processor device 14). The light guide 25 propagates the light from the optical path combining unit 23 to the distal end part 12d of the endoscope 12.

An illumination optical system 30 and an imaging optical system 31 are provided at the distal end part 12d of the endoscope 12. The illumination optical system 30 includes an illumination lens 32, and the observation target is irradiated with the illumination light propagated by the light guide 25 via the illumination lens 32. The imaging optical system 31 includes an objective lens 35 and an imaging sensor 36. Reflected light from the observation target irradiated with the illumination light is incident into the imaging sensor 36 via the objective lens 35. As a result, an image of the observation target is formed on the imaging sensor 36.

The imaging sensor 36 is preferably a color imaging sensor as the imaging sensor 36 that images the observation target. Each pixel of the imaging sensor 36 is provided with any of a blue pixel (B pixel) having a blue (B) color filter, a green pixel (G pixel) having a green (G) color filter, or a red pixel (R pixel) having a red (R) color filter. Spectral transmittances of the B color filter, the G color filter, and the R color filter that determine the spectral sensitivity of the imaging sensor 36 will be described below. For example, the imaging sensor 36 is preferably a color imaging sensor of a Bayer array in which a ratio of the number of pixels of the B pixels, the G pixels, and the R pixels is 1:2:1.

As the imaging sensor 36, a charge coupled device (CCD) imaging sensor or a complementary metal-oxide semiconductor(CMOS) imaging sensor can be used. In addition, a complementary color imaging sensor comprising complementary color filters corresponding to cyan (C), magenta (M), yellow (Y), and green (G) may be used instead of the primary color imaging sensor 36. In a case in which the complementary color imaging sensor is used, image signals corresponding to four colors of C, M, Y, and G are output. Therefore, in a case in which the image signals corresponding to four colors of C, M, Y, and G are converted into image signals corresponding to three colors of R, G, and B by complementary color-primary color conversion, image signals corresponding to the same respective colors of R, G, and B as those of the imaging sensor 36 can be obtained.

The drive of the imaging sensor 36 is controlled by the imaging processor 37. The control of each mode in the imaging processor 37 will be described below. A correlated double sampling/automatic gain control (CDS/AGC) circuit 40 performs correlated double sampling (CDS) or automatic gain control (AGC) on an analog image signal obtained from the imaging sensor 36. The image signal that has passed through the CDS/AGC circuit 40 is converted into a digital image signal by an analog/digital (A/D) converter 41. The digital image signal after the A/D conversion is input to the processor device 14.

The processor device 14 comprises a digital signal processor(DSP) 45, an image processing unit 50, a display control unit 52, and a central control unit 53. In the processor device 14, a program related to various types of processing is incorporated in a program memory (not shown). As the central control unit 53 composed of a processor executes the program in the program memory, functions of the DSP 45, the image processing unit 50, the display control unit 52, and the central control unit 53 are implemented.

The DSP 45 performs various types of signal processing, such as defect correction processing, offset processing, gain correction processing, demosaicing, linear matrix processing, white balance processing, gamma conversion processing, YC conversion processing, and noise reduction processing, on an image signal received from the endoscope 12. In the defect correction processing, a signal of a defective pixel of the imaging sensor 36 is corrected. In the offset processing, a dark current component is removed from the image signal that has passed through the defect correction processing, and an accurate zero level is set. In the gain correction processing, a signal level of each image signal is adjusted by multiplying the image signal of each color after the offset processing by a specific gain. The image signal of each color after the gain correction processing is subjected to the demosaicing and the linear matrix processing for enhancing color reproducibility.

After the linear matrix processing, the white balance processing is performed, and then the brightness and the chroma saturation of each image signal are adjusted through the gamma conversion processing. Thereafter, the YC conversion processing is performed, and a brightness signal Y, a color difference signal Cb, and a color difference signal Cr are output to the DSP 45. The DSP 45 performs the noise reduction processing through, for example, a moving average method or a median filter method.

The image processing unit 50 performs various types of image processing on an image signal from the DSP 45. The image processing includes color conversion processing such as 3×3 matrix processing, gradation transformation processing, and three-dimensional look up table (LUT) processing, color enhancement processing, and structure enhancement processing such as spatial frequency enhancement. The image processing unit 50 performs image processing according to the mode. In the case of the normal observation mode, the image processing unit 50 generates a white light image by performing image processing for a normal observation mode. In the case of the oxygen saturation mode, the image processing unit 50 generates a white light image and transmits an image signal from the DSP 45 to the extended processor device 17 via an image communication unit 51. In the extended processor device 17, an oxygen saturation image is generated based on the transmitted image signal of the endoscope image.

The display control unit 52 performs display control for displaying the white light image NP and image information such as the oxygen saturation image OP, other information, and the like from the image processing unit 50 on the display 15.

The extended processor device 17 receives an image signal from the processor device 14 and performs various types of image processing. The extended processor device 17 calculates oxygen saturation in the oxygen saturation mode and generates an oxygen saturation image OP obtained by visualizing the calculated oxygen saturation. Details of the extended processor device 17 will be described below. The generated oxygen saturation image OP is displayed on the extended display 18.

The function of the extended processor device 17 may be configured to be exerted by the processor device 14, or the function of the extended processor device 17 may be configured to be exerted by both the extended processor device 17 and the processor device 14. In this case, the extended processor device 17 and the processor device 14 may generate different biological parameters. Therefore, as the processor device having the processor that generates the biological parameter image, only the processor device 14, only the extended processor device 17, or both the processor device 14 and the extended processor device 17 can be used.

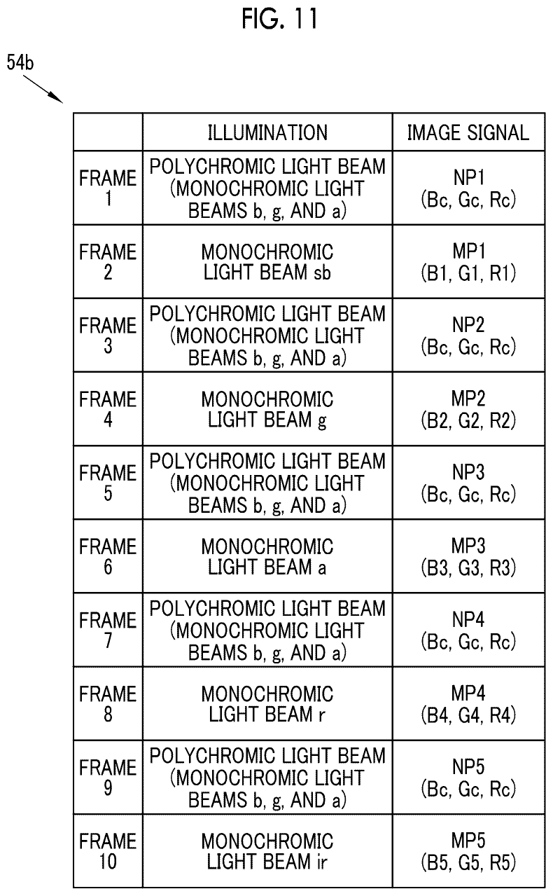

The illumination light in each mode will be described. It is preferable that a central wavelength of each of a plurality of monochromic light beams is any one of 470 nm, 540 nm, 620 nm, 690 nm, or 850 nm. As will be described below, the monochromic light beams having these central wavelengths are light beams in wavelength ranges having different dependencies on the oxygen saturation (see FIG. 11). Among these plurality of monochromic light beams, three or more are used. Preferably, there are five monochromic light beams, and central wavelengths of the five monochromic light beams are 470 nm, 540 nm, 620 nm, 690 nm, and 850 nm. In a case of three monochromic light beams, 540 nm, 620 nm, and 690 nm can be used. Each of these monochromic light beams can be emitted by an LED light source. In each mode, a polychromic light beam, a monochromic light beam, and the like are controlled and emitted by controlling the LED light source to be turned on or off.

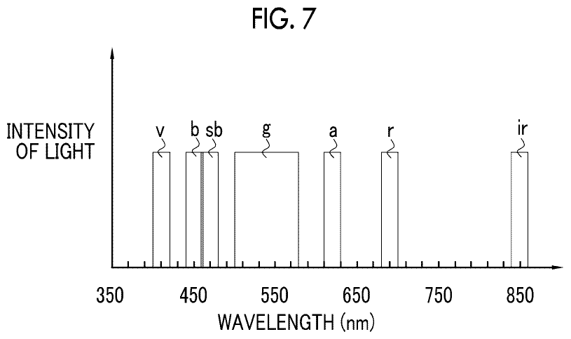

As shown in FIG. 7, the v-LED 20g emits a monochromic light beam v having a central wavelength of 410 nm. The b-LED 20a emits a monochromic light beam b having a central wavelength of 450 nm. The sb-LED 20b emits a monochromic light beam sb having a central wavelength of 470 nm. The g-LED 20c emits a monochromic light beam g having a central wavelength of 540 nm. The a-LED 20d emits a monochromic light beam a having a central wavelength of 620 nm. The r-LED 20e emits a monochromic light beam r having a central wavelength of 690 nm. The ir-LED 20f emits a monochromic light beam ir having a central wavelength of 850 nm. The central wavelength of each monochromic light beam may be the same as or different from a peak wavelength. In addition, in the drawing, a graph of intensity of light at each wavelength is schematically shown and does not necessarily indicate actual intensity of light.

It is preferable that the polychromic light beam is generated by emitting two or more monochromic light beams among the plurality of monochromic light beams. In the normal observation mode, as the v-LED 20g, the b-LED 20a, the g-LED 20c, and the r-LED 20e are simultaneously turned on, a polychromic light beam including the monochromic light beam v having a central wavelength of 410 nm, the monochromic light beam b having a central wavelength of 450 nm, the monochromic light beam g having a central wavelength of 540 nm, and the monochromic light beam r having a central wavelength of 690 nm are emitted.

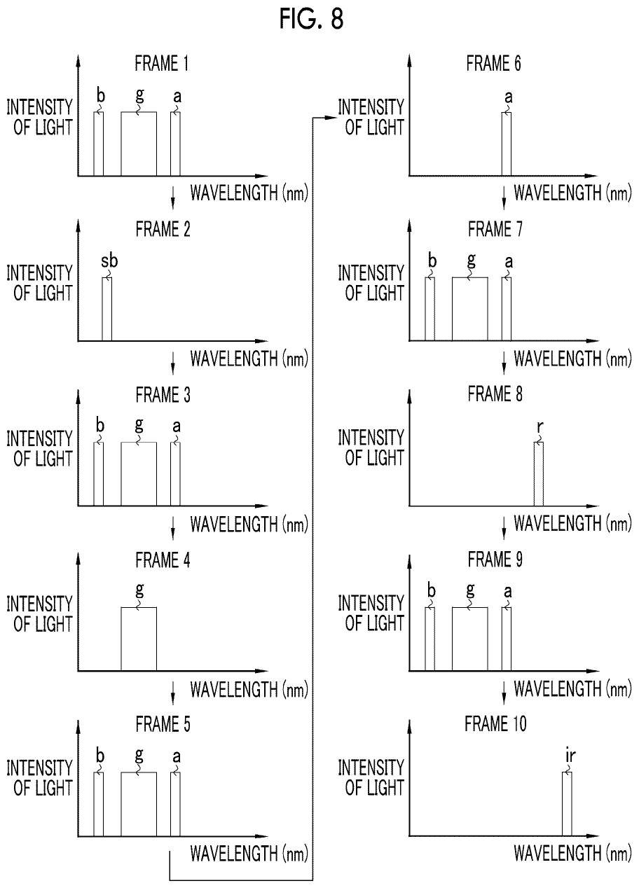

As shown in FIG. 8, in the oxygen saturation mode, monochromic light beams having central wavelengths of 450 nm, 470 nm, 540 nm, 620 nm, 690 nm, and 850 nm are emitted in order in even-numbered frames of a frame 2, a frame 4, a frame 6, a frame 8, and a frame 10, and a polychromic light beam is emitted between the light emissions of the respective monochromic light beams. The polychromic light beam in the oxygen saturation mode is a polychromic light beam including the monochromic light beam b having a central wavelength of 450 nm, the monochromic light beam g having a central wavelength of 540 nm, and the monochromic light beam r having a central wavelength of 690 nm, and are emitted in odd-numbered frames of a frame 1, a frame 3, a frame 5, a frame 7, and a frame 9. As described above, it is preferable that the light source device 13 illuminates the observation target with light-emitting units that emit all of the plurality of monochromic light beams in ascending order of the central wavelength, and it is preferable that the light source device 13 illuminates the observation target with light-emitting units that emit all of the plurality of monochromic light beams in a preset order. Further, it is preferable that the light source device 13 alternately emits the polychromic light beam and any of the plurality of monochromic light beams for each frame.

Therefore, in a case in which there are five monochromic light beams, central wavelengths of the five monochromic light beams are 470 nm, 540 nm, 620 nm, 690 nm, and 850 nm, and the polychromic light beam and the monochromic light beams are alternately emitted for each frame, the light emission for 10 frames of the frames 1 to 10 is repeatedly performed as shown in FIG. 8. In this case, since the frames 1 to 10 are repeated, the frames 1 to 10 are light-emitting units. In addition, one light-emitting unit is 10 frames of the frames 1 to 10, and 10 endoscope images are acquired in one light-emitting unit.

Specifically, in a case in which the light emission is repeatedly performed using the light-emitting units for 10 frames of the frames 1 to 10, the light source device 13 emits, as illumination light, monochromic light beams sb, g, a, r, and ir having central wavelengths of 470 nm, 540 nm, 620 nm, 690 nm, and 850 nm, respectively, in frames 2, 4, 6, 8, and 10 of even-numbered frames, and a polychromic light beam including a monochromic light beam b having a central wavelength of 450 nm, a monochromic light beam g having a central wavelength of 540 nm, and a monochromic light beam r having a central wavelength of 690 nm in odd-numbered frames 1, 3, 5, 7, and 9 between the even-numbered frames.

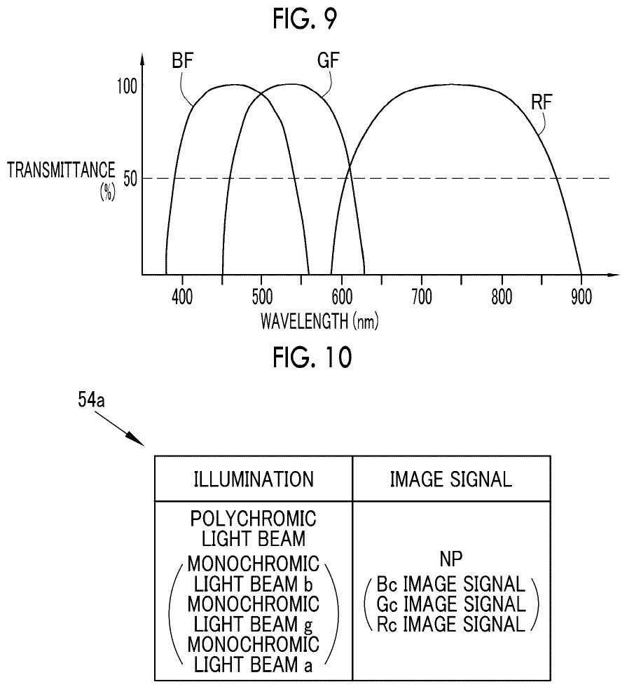

The spectral transmittances of the B color filter, the G color filter, and the R color filter will be described. As shown in FIG. 9, a B color filter BF provided in the B pixel of the imaging sensor 36 mainly transmits light in a blue band, specifically, light in a wavelength range of 380 to 560 nm (blue transmission band). A peak wavelength at which a transmittance is maximized exists around 460 to 470 nm. A G color filter GF provided in the G pixel of the imaging sensor 36 mainly transmits light in a green band, specifically, light having a wavelength range of 450 to 630 nm (green transmission band). An R color filter RF provided in the R pixel of the imaging sensor 36 mainly transmits light in the red band, specifically, light of 580 to 900 nm (red transmission band).

The control of each mode in the imaging processor 37 will be described. As shown in FIG. 10, in a normal observation mode 54a, the imaging processor 37 controls the imaging sensor 36 to image the observation target under illumination with the polychromic light beam including the monochromic light beam b, the monochromic light beam g, and the monochromic light beam r for each frame. Then, the illumination light which is a polychromic light beam is continuously emitted to continue the imaging for each frame. As a result, a Bc image signal is output from the B pixel of the imaging sensor 36, a Gc image signal is output from the G pixel, and an Rc image signal is output from the R pixel. The white light image NP is generated based on the Bc image signal, the Gc image signal, and the Rc image signal. Since the white light image NP is an endoscope image obtained by imaging the observation target illuminated with a polychromic light beam emitting a plurality of monochromic light beams simultaneously, the white light image NP is a polychromic image.

As shown in FIG. 11, in an oxygen saturation mode 54b, in frames 1, 3, 5, 7, and 9 of odd-numbered frames, the imaging processor 37 performs control of imaging the observation target under illumination with the polychromic light beam including the monochromic light beam b, the monochromic light beam g, and the monochromic light beam r. As a result, in the frames 1, 3, 5, 7, and 9, a Bc image signal is output from the B pixel of the imaging sensor 36, a Gc image signal is output from the G pixel, and an Rc image signal is output from the R pixel. The first to fifth white light images NP1, NP2, NP3, NP4, and NP5 are generated in the odd-numbered frames 1, 3, 5, 7, and 9 based on the Bc image signal, the Gc image signal, and the Rc image signal.

On the other hand, in the frame 2 of the even-numbered frame, the imaging processor 37 performs control of imaging the observation target under illumination with the monochromic light beam sb. As a result, a B1 image signal is output from the B pixel of the imaging sensor 36, a G1 image signal is output from the G pixel, and an R1 image signal is output from the R pixel. Similarly, in the frames 4, 6, 8, and 10 of the even-numbered frames, the imaging processor 37 performs control of imaging the observation target under illumination with the monochromic light beams g, a, r, and ir. As a result, B2, B3, B4, and B5 image signals are output from the B pixel of the imaging sensor 36, G2, G3, G4, and G5 image signals are output from the G pixel, and R2, R3, R4, and R5 image signals are output from the R pixel.

The first monochromic image MP1 is configured by the B1 image signal, the G1 image signal, and the R1 image signal. In addition, the second monochromic image MP2 is configured by the B2 image signal, the G2 image signal, and the R2 image signal. In addition, the third monochromic image MP3 is configured by the B3 image signal, the G3 image signal, and the R3 image signal. In addition, the fourth monochromic image MP4 is configured by the B4 image signal, the G4 image signal, and the R4 image signal. In addition, the fifth monochromic image MP5 is configured by the B5 image signal, the G5 image signal, and the R5 image signal.

In the calculation of the biological parameter such as the oxygen saturation, the B1 image signal, the B2 image signal, the G2 image signal, the R3 image signal, the R4 image signal, the B5 image signal, the G5 image signal, and the R5 image signal among the image signals obtained in the frames 2, 4, 6, 8, and 10 of the even-numbered frames are used. For the B5 image signal, the G5 image signal, and the R5 image signal, an MR5 image signal obtained by synthesizing (adding together) the three image signals is used. Hereinafter, in a case of distinguishing from a standard image described below, the B1 image signal is referred to as a B1_{img} image signal, the B2 image signal is referred to as a B2_{img} image signal, the G2 image signal is referred to as a G2_{img} image signal, the R3 image signal is referred to as a G3_{img} image signal, the R3 image signal is referred to as an R3_{img} image signal, the R4 image signal is referred to as an R4_{img} image signal, and the MR5 image signal is referred to as an MR5_{img} image signal. The B1_{img} image signal, the B2_{img} image signal, the G2_{img} image signal, the R3_{img} image signal, the R4_{img} image signal, the B5_{img} image signal, the G5_{img} image signal, and the MR5_{img} image signal are indicated including any of a state before registration or a state after registration.

The B1 image signal includes image information related to the monochromic light beam sb in the light transmitted through the B color filter BF. The B2 image signal includes image information related to the monochromic light beam g in the light transmitted through the B color filter BF. The G2 image signal includes image information related to the monochromic light beam g in the light transmitted through the G color filter GF. The R3 image signal includes image information related to the monochromic light beam a in the light transmitted through the R color filter RF. The R4 image signal includes image information related to the monochromic light beam r in the light transmitted through the R color filter. The B5 image signal includes image information related to the monochromic light beam ir in the light transmitted through the B color filter. The G5 image signal includes image information related to the monochromic light beam ir in the light transmitted through the G color filter. The R5 image signal includes image information related to the monochromic light beam ir in the light transmitted through the R color filter.

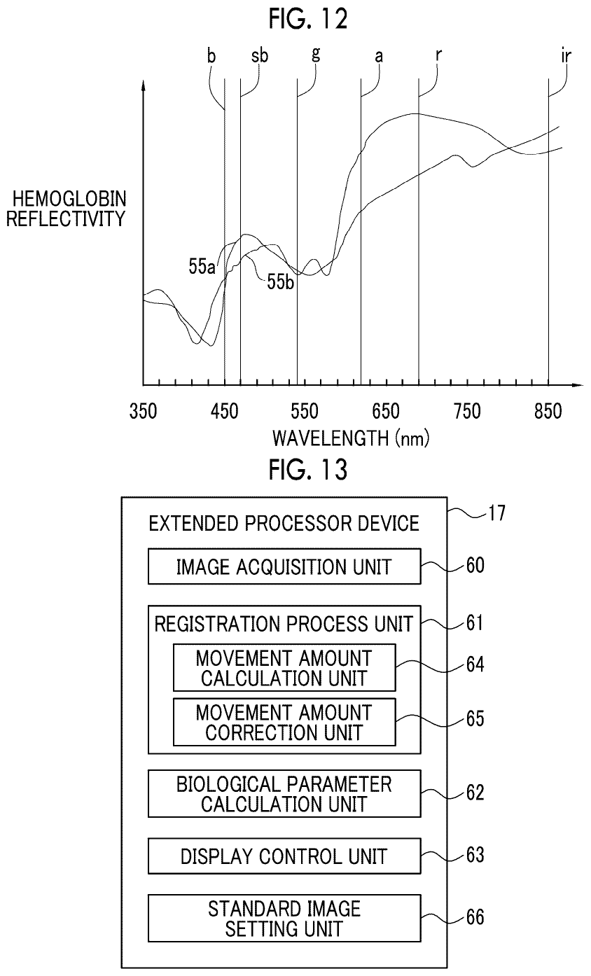

In addition, the image information related to the monochromic light beam sb includes, as shown in FIG. 12, image information of a wavelength range sb whose reflection spectrum changes due to a change in oxygen saturation of hemoglobin in the blood. Similarly, the image information related to the monochromic light beams g, a, r, and ir includes image information of the wavelength ranges g, a, r, and ir whose reflection spectra change due to a change in oxygen saturation of hemoglobin in the blood. A curve 55a represents a reflection spectrum of reduced hemoglobin, and a curve 55b represents a reflection spectrum of oxidized hemoglobin.

The extended processor device 17 will be described. As shown in FIG. 13, the extended processor device 17 comprises an image acquisition unit 60, a registration process unit 61, a biological parameter calculation unit 62, a display control unit 63, and a standard image setting unit 66. The registration process unit 61 includes a movement amount calculation unit 64 and a movement amount correction unit 65. In the extended processor device 17, a program related to various types of processing is incorporated in a program memory (not shown). As a central control unit (not shown) composed of a processor executes the program in the program memory, functions of the image acquisition unit 60, the registration process unit 61, the biological parameter calculation unit 62, and the display control unit 63 are implemented.

The image acquisition unit 60 acquires, via the processor device 14, a polychromic image and a monochromic image obtained by imaging the observation target illuminated with a polychromic light beam and a monochromic light beam by means of the imaging sensor 36. The processor calculates the oxygen saturation by performing processing based on the monochromic image. In this case, as described above, the registration process is performed on the monochromic image. The registration process includes a movement calculation process of the movement amount calculation unit 64 and a movement correction process of the movement amount correction unit 65. The motion calculation process is a process of calculating a movement amount of the monochromic image based on a plurality of polychromic images, and the movement correction process is a process of generating an aligned monochromic image by performing correction based on the movement amount on the monochromic image of which the movement amount is calculated.

The movement amount calculation unit 64 included in the registration process unit 61 calculates the movement amount by estimating the movement amount for each monochromic image, and generates a movement amount-containing monochromic image which is a monochromic image to which the movement amount is added (see FIG. 1). In addition, the movement amount correction unit 65 performs the registration by correcting each movement amount-containing monochromic image based on the movement amount calculated by the movement amount calculation unit 64. These processes can be performed by a known method, for example, a method described in JP2016-185398A. In the present embodiment, the registration is performed by calculating a movement amount of an image in a horizontal direction, calculating a movement amount of the image in a vertical direction, and correcting the movement amounts. Therefore, the registration includes correcting the movement amount in the horizontal direction, correcting the movement amount in the vertical direction, and combining these movement amounts.

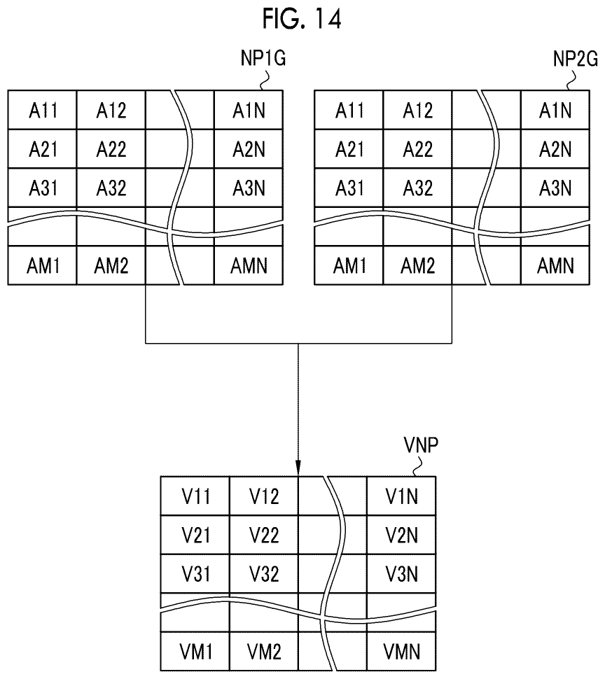

The calculation of the movement amount of the image in the horizontal direction is performed as follows. Specifically, in a case in which the movement amount in the horizontal direction is calculated using a first polychromic image NP1G and a second polychromic image NP2G, as shown in FIG. 14, two images of the first polychromic image NP1G and the second polychromic image NP2G, for which the relative movement amount is calculated, are divided into a plurality of predetermined regions (M×N rectangular regions Mij (i=1, . . . , M, j=1, . . . , N) of M vertical lines and N horizontal lines), and block matching is performed on small regions Mij at the same position of each image to calculate a representative movement vector Vij of the small region. The first polychromic image NP1G and the second polychromic image NP2G are G channel images. The movement vector Vij is associated with a central pixel of each small region Mij, and the movement vectors of the other pixels are obtained by linear interpolation in consideration of a representative movement vector of a peripheral small region (vector diagram VNP). This operation is performed using a G channel image in which a blood vessel is shown with high contrast. This is because the movement amount can be accurately calculated by the blood vessel shown in the image.

After acquiring the movement amount as a vector based on the polychromic images, the movement amount of the monochromic image acquired between two polychromic images is estimated. In the estimation, a half of a sum of the vectors of the movement amounts obtained based on the two polychromic images can be estimated as the movement amount of the monochromic image. This is because the monochromic image is acquired at an exact midpoint in time between the acquisition times of the two polychromic images. As described above, the movement amount of the monochromic image is obtained by interpolating the movement amounts of the two polychromic images and, for example, performing linear interpolation to acquire the movement amount of the monochromic image as a vector. The movement amount is acquired for each monochromic image to form the movement amount-containing monochromic image, and the registration of each monochromic image in the horizontal direction is performed based on the movement amount. The above-described registration in the horizontal direction is performed for the first to fifth monochromic images MP1 to MP5.

Therefore, it is preferable that the movement amount calculation process of the movement amount calculation unit 64 is a process of calculating the movement amount of the monochromic image based on two polychromic images obtained in the immediately preceding and immediately following frames of the monochromic image.

Next, regarding calculation of a movement amount in a direction perpendicular to an image, in a case in which there is a movement in which a distance to the observation target changes between the frames, the illuminance changes between the frames, and a spectral signal ratio between the frames changes from a true value because of an influence of the change, resulting in noise in the oxygen saturation to be calculated. Therefore, the illuminance change between the frames is corrected by the following procedure.

A predetermined region is set in the center of each image (using an R channel image) of the odd-numbered frames, and average pixel values in the regions are obtained (r(i), i=1, 3, . . . , 11). Using these, an estimated value of the value in the even-numbered frame is obtained by averaging the adjacent odd-numbered frames (r(2)=(r(1)+r(3))/2, . . . ). Using these values r(2), r(4), . . . , of the even-numbered frames, the entire pixel values of the even-numbered frame images (frames 2, 4, 8, and 10, see FIG. 11) after the registration in the horizontal direction are multiplied by r(6)/r(2), r(6)/r(4), r(6)/r(8), and r(6)/r(10). In this way, the illuminance change between the frames can be corrected with the frame 6 as a reference.

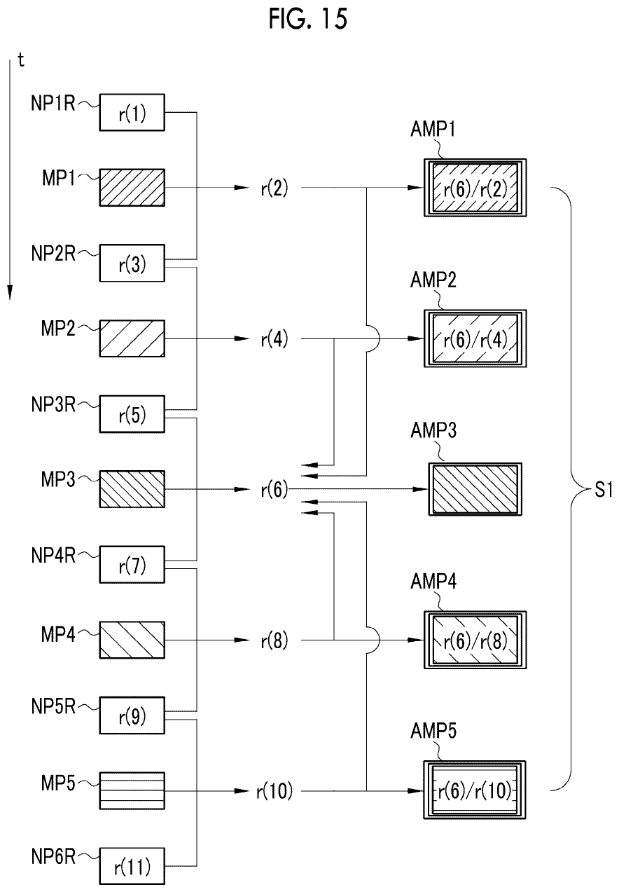

As shown in FIG. 15, a predetermined region is set in the center of a first polychromic image NP1R and a second polychromic image NP2R of the odd-numbered frames, and average pixel values in the regions are r(1) and r(3). The first polychromic image NP1R and the second polychromic image NP2R are R channel images. For the first monochromic image MP1 of the even-numbered frame acquired between the first polychromic image NP1R and the second polychromic image NP2R, the entire pixel values of the frame image (see FIG. 14) that has been subjected to the registration in the horizontal direction are multiplied by r(6)/r(2), thereby obtaining an aligned first monochromic image AMP1. As a result, using the third monochromic image MP3 of the frame 6 as a reference, it is possible to perform correction for the registration based on the movement amount in the direction perpendicular to the image, on the first monochromic image MP1 after the registration in the horizontal direction.

Similarly, for the second monochromic image MP2, the entire pixel values of the frame image (see FIG. 14) that has been subjected to the registration in the horizontal direction are multiplied by r(6)/r(4), thereby obtaining an aligned second monochromic image AMP2. Since the third monochromic image MP3 is a reference, an aligned third monochromic image AMP3 is obtained without any multiplication here. Similarly, for the fourth monochromic image MP4, the entire pixel values of the frame image (see FIG. 14) that has been subjected to the registration in the horizontal direction are multiplied by r(6)/r(8), thereby obtaining an aligned fourth monochromic image AMP4. Similarly, for the fifth monochromic image MP5, the entire pixel values of the frame image (see FIG. 14) that has been subjected to the registration in the horizontal direction are multiplied by r(6)/r(10), thereby obtaining an aligned fifth monochromic image AMP5.

Since the aligned first monochromic image AMP1 to the aligned fifth monochromic image AMP5 are images obtained in one light-emitting unit, these are regarded as an aligned monochromic image set. In the oxygen saturation mode, as the user continues the observation, a time-series aligned monochromic image set is obtained one after another.

The registration process unit 61 may determine a degree of the misregistration in a case in which each monochromic image is registered, in a case of generating the aligned monochromic image set. That is, the movement amount calculation unit 64 calculates a total movement amount obtained by adding up the movement amounts between the monochromic images acquired in the light-emitting units, for each light-emitting unit. Then, the calculated total movement amount may be regarded as the degree of the misregistration, and it may be determined whether the misregistration exceeds a predetermined threshold value. The determination of the misregistration between the monochromic images acquired in the light-emitting units can be performed by adding up the movement vectors between all the monochromic images and comparing the total with a preset threshold value. In addition, only the magnitudes of the movement vectors may be added up and used as the threshold value, or a specific direction may be used as the threshold value.

In a case in which the total movement amount is within a preset range, an alignment image set including a plurality of the aligned monochromic images obtained in the light-emitting units may be acquired. As a result, in a case in which the accuracy, reliability, and the like of the calculated oxygen saturation are low, such as a case in which the misregistration is large, the oxygen saturation image is not displayed on the extended display 18. Therefore, the reliability of the displayed oxygen saturation image is improved.

In a case in which the misregistration between the monochromic images acquired in the light-emitting units exceeds a predetermined threshold value, a resolution may be adjusted in each monochromic image according to a degree of the misregistration, and then the monochromic images may be used as the aligned monochromic image set. The resolution adjustment processing is performed by, for example, an average reduction of the images. In this case, as the adjustment of the resolution, a degree to which the resolution is reduced can be set to an average reduction of ⅛ in a case in which it is determined that the degree of the misregistration is small, and the degree of reduction in the resolution can be set to an average reduction 1/16 in a case in which it is determined that the degree of the misregistration is large. The resolution of each monochromic image is reduced, and then the aligned monochromic image set is generated. In addition, it is preferable that the aligned monochromic image set with the reduced resolution is restored to the original resolution after the oxygen saturation level is calculated by the biological parameter calculation unit 62, and then an oxygen saturation image is generated.

The biological parameter calculation unit 62 calculates the biological parameter such that an error calculation value based on an error between the standard image and the aligned monochromic image is within a specific range (fitting process). Since the standard image is determined based on a spectral model that uses the biological parameter as an argument, the standard image is defined as a function of the biological parameter. In the present embodiment, as the biological parameters, four biological parameters, that is, a hemoglobin concentration Hb, a bilirubin concentration Bb, and a scattering wavelength-dependent parameter b are used in addition to oxygen saturation S.

The standard image is determined for each aligned monochromic image. In the present embodiment, a B1_{std} image signal, a B2_{std} image signal, a G2_{std} image signal, an R3_{std} image signal, an R4_{std} image signal, and an MR5_{std} image signals are determined as standard images corresponding to the B1_{img} image signal, the B2_{img} image signal, the G2_{img} image signal, the R3_{img} image signal, the R4_{img} image signal, and the MR5_{img} image signal.

It is preferable that an error calculation value Diff is calculated by a square error calculation equation based on (Equation 1).

$$Diff = {{W}_{B 1} ( B {1}_{std} ( S , Hb , Bb , b ) - B {1}_{img} )}^{2} + {{W}_{B 2} ( B {2}_{std} ( S , Hb , Bb , b ) - B {2}_{img} )}^{2} + {{W}_{G 1} ( G {2}_{std} ( S , Hb , Bb , b ) - G {2}_{img} )}^{2} + {{W}_{R 3} ( R {3}_{std} ( S , Hb , Bb , b ) - R {3}_{img} )}^{2} + {{W}_{R 4} ( R {4}_{std} ( S , Hb , Bb , b ) - R {4}_{img} )}^{2} + {{W}_{MR 5} ( MR {5}_{std} ( S , Hb , Bb , b ) - MR {5}_{img} )}^{2} \qquad ( Equation 1 )$$

Note that B1_{std}(S, Hb, Bb, b), B2_{std}(S, Hb, Bb, b), G2_{std}(S, Hb, Bb, b), R3_{std}(S, Hb, Bb, b), R4_{std}(S, Hb, Bb, b), and MR5_{std}(S, Hb, Bb, b) represent a B1_{std} image signal, a B2_{std} image signal, a G2_{std} image signal, an R3_{std} image signal, an R4_{std} image signal, and an MR5_{std} image signal, respectively, and also represent functions of the oxygen saturation S, the hemoglobin concentration Hb, the bilirubin concentration Bb, and the scattering wavelength-dependent parameter b. In addition, B1_{img}, B2_{img}, G2_{img}, R3_{img}, R4_{img}, and MR5_{img} represent a B1_{img} image signal, a B2_{img} image signal, a G2_{img} image signal, an R3_{img} image signal, an R4_{img} image signal, and an MR5_{img} image signal, respectively. In addition, W_{B1}, W_{B2}, W_{G2}, W_{R3}, W_{R4}, and W_{MR5} represent weighting factors for a squared difference value of each term.

In a case in which the error calculation value Diff is calculated by Equation (1), the biological parameter calculation unit 62 calculates the biological parameter such that the error calculation value Diff is minimized. As a result, the oxygen saturation S, the hemoglobin concentration Hb, the bilirubin concentration Bb, and the scattering wavelength-dependent parameter b are obtained as the biological parameters. The biological parameter may be calculated for each pixel or may be calculated for each pixel region having a plurality of pixels.

In the present embodiment, it is preferable that the biological parameter is calculated using N or more (N is an integer greater than 3) standard spectral signals at different wavelengths decided by N−1 biological parameters including oxygen saturation and N or more first spectral signals corresponding to the wavelengths of the N or more standard signals, the N or more standard spectral signals. By calculating the biological parameter using N first spectral signals larger than N−1, which is the number of the biological parameters, it is possible to calculate the N−1 biological parameters with high accuracy.

In the present embodiment, in order to calculate four biological parameters, that is, the oxygen saturation S, the hemoglobin concentration Hb, the bilirubin concentration Bb, and the scattering wavelength-dependent parameter b with high accuracy, six image signals, that is, the B1 image signal, the B2 image signal, the G2 image signal, the R3 image signal, the R4 image signal, and the MR5 image signal are used as measured first spectral image signals.

The display control unit 63 displays the oxygen saturation image obtained by visualizing the calculated oxygen saturation on the extended display 18 in various display aspects. For example, it is preferable that a still image of the oxygen saturation is displayed in parallel with a video of the white light image obtained using the polychromic light beam on the extended display 18. In addition, in a case in which the video of the white light image is displayed in parallel with a video of the oxygen saturation image, it is preferable to perform the display by reducing a frame rate of the video of the oxygen saturation image. As described above, it is preferable that the white light image is generated based on a polychromic image obtained by imaging the observation target illuminated with a polychromic light beam by means of the imaging sensor 36. It is preferable that the polychromic image is the Bc image signal, the Gc image signal, and the Rc image signal obtained in odd-numbered frames (frame 1, 3, 5, 7, and 9) in the oxygen saturation mode.

The control of displaying the oxygen saturation image may be performed based on switching of the observation mode. In a case in which the user switches a mode from the normal observation mode to the biological parameter image acquisition mode by operating the mode selector switch 12f, which is a switching operation unit, the oxygen saturation image, which is a biological parameter image, may be generated based on the aligned monochromic image set acquired first after the switching, and control of displaying the generated oxygen saturation image on the extended display 18 may be performed. In a case in which the oxygen saturation image is displayed on the extended display 18, the observation mode may be automatically restored to the normal observation mode. In addition, in this case, the oxygen saturation image may remain displayed on the extended display 18 until the next oxygen saturation image is generated.

In addition to the oxygen saturation, a hemoglobin concentration, a bilirubin concentration, and a scattering wavelength-dependent parameter are calculated as the biological parameters, and these may also be displayed as images on the extended display 18. For example, a hemoglobin concentration image based on the hemoglobin concentration may be displayed on the extended display 18. Since a combination of the hemoglobin concentration and the oxygen saturation can be used as an indicator of congestion, it is preferable to display the oxygen saturation image and the hemoglobin concentration image on the extended display 18 in parallel with the white light image. In addition, an indicator combining the oxygen saturation and the hemoglobin concentration may be visualized and displayed on the extended display 18. For example, a pseudo-color image in which the oxygen saturation is assigned to color differences Cr and Cb and the hemoglobin concentration is assigned to a brightness Y is displayed on the extended display 18. In this case, in the pseudo-color image, the higher the hemoglobin concentration, the darker the display.

The standard image setting unit 66 sets a standard image based on a light emission spectrum of the illumination light, a spectral reflectivity of a living body determined from the spectral model, and spectral sensitivity of the imaging sensor 36. It is preferable that the standard image is standardized with a standard light emission value corresponding to the light emission spectrum.

In the present embodiment, B1_{std}(S, Hb, Bb, b) is set according to (Equation 2).

$$B {1}_{std} ( S , Hb , Bb , b ) = \frac{\int Lsb ( λ ) R ( λ ; S , Hb , Bb , b ) Sb ( λ ) d λ}{\int Lsb ( λ ) d λ} \qquad ( Equation 2 )$$

In (Equation 2), “Lsb(λ)” represents a brightness of the monochromic light beam sb. “R(λ; S, Hb, Bb, b)” represents a spectral reflectivity of a living body determined from the spectral model. “Sb(λ)” represents sensitivity of the B pixel of the imaging sensor 36.

The denominator of (Equation 2) is a standard light emission value corresponding to the light emission spectrum of the monochromic light beam sb, and B1_{std}(S, Hb, Bb, b) is standardized with the standard light emission value.

In the present embodiment, B2_{std}(S, Hb, Bb, b) is set according to (Equation 3).

$$B {2}_{std} ( S , Hb , Bb , b ) = \frac{\int Lg ( λ ) R ( λ ; S , Hb , Bb , b ) Sb ( λ ) d λ}{\int Lg ( λ ) d λ} \qquad ( Equation 3 )$$

In (Equation 3), “Lg(λ)” represents a brightness of the monochromic light beam g. “R(λ; S, Hb, Bb, b)” represents a spectral reflectivity of a living body determined from the spectral model. “Sb(λ)” represents sensitivity of the B pixel of the imaging sensor 36.

The denominator of (Equation 3) is a standard light emission value corresponding to the light emission spectrum of the monochromic light beam g, and B2_{std}(S, Hb, Bb, b) is standardized with the standard light emission value.

In the present embodiment, G2_{std}(S, Hb, Bb, b) is set according to (Equation 4).

$$G {2}_{std} ( S , Hb , Bb , b ) = \frac{\int Lg ( λ ) R ( λ ; S , Hb , Bb , b ) Sg ( λ ) d λ}{\int Lg ( λ ) d λ} \qquad ( Equation 4 )$$

In (Equation 4), “Lg(λ)” represents a brightness of the monochromic light beam g. “R(λ; S, Hb, Bb, b)” represents a spectral reflectivity of a living body determined from the spectral model. “Sg(λ)” represents sensitivity of the G pixel of the imaging sensor 36.

The denominator of (Equation 4) is a standard light emission value corresponding to the light emission spectrum of the monochromic light beam g, and G2_{std}(S, Hb, Bb, b) is standardized with the standard light emission value.

In the present embodiment, R3_{std}(S, Hb, Bb, b) is set according to (Equation 5).

$$R {3}_{std} ( S , Hb , Bb , b ) = \frac{\int La ( λ ) R ( λ ; S , Hb , Bb , b ) Sr ( λ ) d λ}{\int La ( λ ) d λ} \qquad ( Equation 5 )$$

In (Equation 5), “La(λ)” represents a brightness of the monochromic light beam a. “R(λ; S, Hb, Bb, b)” represents a spectral reflectivity of a living body determined from the spectral model. “Sr(λ)” represents sensitivity of the R pixel of the imaging sensor 36.

The denominator of (Equation 5) is a standard light emission value corresponding to the light emission spectrum of the monochromic light beam a, and R3_{std}(S, Hb, Bb, b) is standardized with the standard light emission value.

In the present embodiment, R4_{std}(S, Hb, Bb, b) is set according to (Equation 6).

$$R {4}_{std} ( S , Hb , Bb , b ) = \frac{\int Lr ( λ ) R ( λ ; S , Hb , Bb , b ) Sr ( λ ) d λ}{\int Lr ( λ ) d λ} \qquad ( Equation 6 )$$

In (Equation 6), “Lr(λ)” represents a brightness of the monochromic light beam r. “R(λ; S, Hb, Bb, b)” represents a spectral reflectivity of a living body determined from the spectral model. “Sr(λ)” represents sensitivity of the R pixel of the imaging sensor 36.

The denominator of (Equation 6) is a standard light emission value corresponding to the light emission spectrum of the monochromic light beam r, and R4_{std}(S, Hb, Bb, b) is standardized with the standard light emission value.

In the present embodiment, MR5_{std}(S, Hb, Bb, b) is set according to (Equation 7).

$$R {5}_{std} ( S , Hb , Bb , b ) = \frac{\int Lir ( λ ) R ( λ ; S , Hb , Bb , b ) Sr ( λ ) d λ + \int Lir ( λ ) R ( λ ; S , Hb , Bb , b ) Sg ( λ ) d λ + \int Lir ( λ ) R ( λ ; S , Hb , Bb , b ) Sb ( λ ) d λ}{\int Lir ( λ ) d λ} \qquad ( Equation 7 )$$

In (Equation 7), “Lir(λ)” represents a brightness of the monochromic light beam r. “R(λ; S, Hb, Bb, b)” represents a spectral reflectivity of a living body determined from the spectral model. “Sr(λ)”, “Sg(λ)”, and “Sb(λ)” represent sensitivity of the R pixel, sensitivity of the G pixel, and sensitivity of the B pixel of the imaging sensor 36, respectively.

The denominator of (Equation 7) is a standard light emission value corresponding to the light emission spectrum of the monochromic light beam ir, and MR5_{std}(S, Hb, Bb, b) is standardized with the standard light emission value.

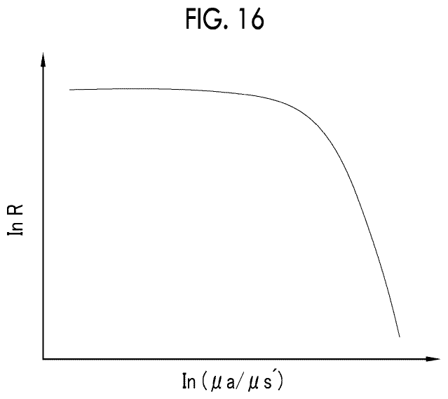

The spectral reflectivity R(λ; S, Hb, Bb, b) of the living body is calculated from a ratio μa/μs' of an absorption coefficient μa(λ; Hb, S, Bb) to a scattering coefficient μs′(λ; b). A relationship between the spectral reflectivity R and the ratio μa/μs′ is shown in FIG. 16. The spectral reflectivity R (ratio μa/μs′) may be stored in a table in a functional form, or may be approximated and held by a simple function such as a polynomial. In addition, in FIG. 16, both the spectral reflectivity R and the ratio μa/μs′ are logarithmic.

The absorption coefficient μa(λ; Hb, S, Bb) is represented by (Equation 8).

$$μa ( λ ; Hb , S , Bb ) = Hb \times \{ S / 100 \times μ aHb O 2 ( λ ) + ( 1 - S / 100 ) \times μ aHb ( λ ) \} + Bb \times μ aBb ( λ ) \qquad ( Equation 8 )$$

In (Equation 8), μaHbO2 (λ) represents an absorption coefficient of oxygenated hemoglobin, and Hb (2) represents an absorption coefficient of reduced hemoglobin. μaBb(λ) represents an absorption coefficient of bilirubin. λ represents a wavelength (the same applies to Equation (9))

In addition, the scattering coefficient μs′ (λ; b) is represented by (Equation 9).

$$μs ’ ( λ ; b ) = 14 {cm}^{- 1} \times {( λ / 500 nm )}^{- b} \qquad ( Equation 9 )$$

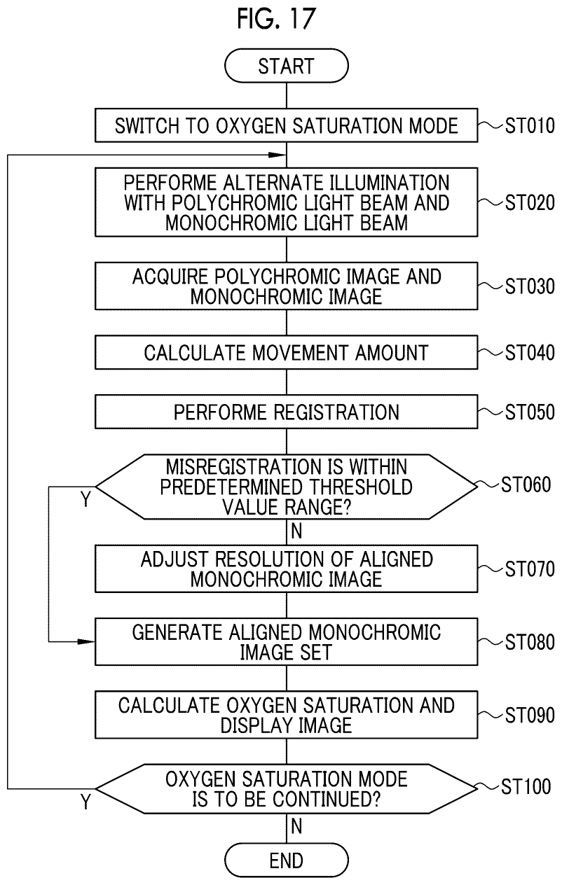

Next, a series of flows of the oxygen saturation mode will be described with reference to the flowchart of FIG. 17. In a case in which the user is observing the observation target in the normal observation mode, the user operates the mode selector switch 12f to switch to observation in the oxygen saturation mode (step ST010). As the illumination light, alternate illumination with a polychromic light beam and a monochromic light beam is performed (step ST020). The image acquisition unit 60 acquires a polychromic image and a monochromic image obtained by imaging the observation target illuminated with the polychromic light beam and the observation target illuminated with the monochromic light beam by means of the imaging sensor 36 (step ST030).

The registration process unit 61 calculates the movement amount for the monochromic image using the polychromic image (step ST040). The movement amount is calculated for a plurality of monochromic images obtained in the light-emitting units, and the registration is performed based on the calculated movement amount (step ST050).

In a case of performing the registration, the registration process unit 61 may determine whether the misregistration is within a predetermined threshold value in a plurality of aligned monochromic images obtained in the light-emitting units. In a case in which the misregistration is within the threshold value range (Y in step ST060), an aligned monochromic image set is generated (step ST080). In a case in which the misregistration is out of the threshold value range (N in step ST060), the resolution is adjusted in the plurality of aligned monochromic images (step ST070). The adjustment of the resolution may be performed according to the degree of the misregistration. For example, the average reduction of the images is performed, and then the aligned monochromic image set is generated (step ST080). The average reduction of the images is set to ⅛ in a case in which it is determined that the degree of the misregistration is medium, and is set to 1/16 in a case in which it is determined that the degree of the misregistration is large.

The oxygen saturation is calculated for each pixel of the aligned monochromic image in the generated aligned monochromic image set, and the oxygen saturation image is generated based on the calculated oxygen saturation and displayed on the extended display 18 (step ST090). In a case in which the oxygen saturation mode is to be continued (Y in step ST100), the observation with the specific illumination light is continued. In a case in which the oxygen saturation mode is to be ended (N in step ST100), the oxygen saturation mode is ended.

### Second Embodiment

In a second embodiment, in a case of calculating the biological parameters, the biological parameters are not used as an argument (variable) in the spectral model, but are instead determined in advance as a plurality of fixed values, and a biological parameter satisfying a condition is calculated as the biological parameter from among the biological parameters as the fixed values. In the second embodiment, M sets of standard images used for calculating the biological parameters are set for each spectral model based on the light emission spectrum of the illumination light, the spectral reflectivity of the living body determined from the spectral model, and the spectral sensitivity of the imaging sensor 36. The procedure other than the setting of the standard image is the same as that of the first embodiment.

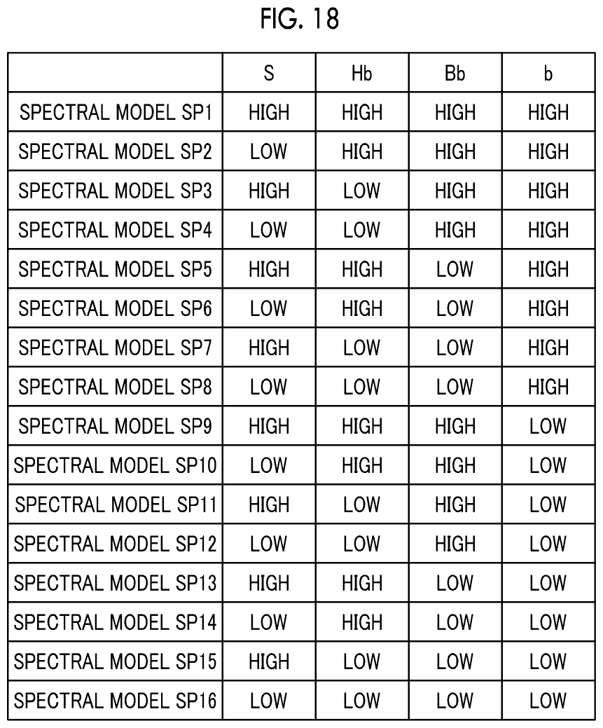

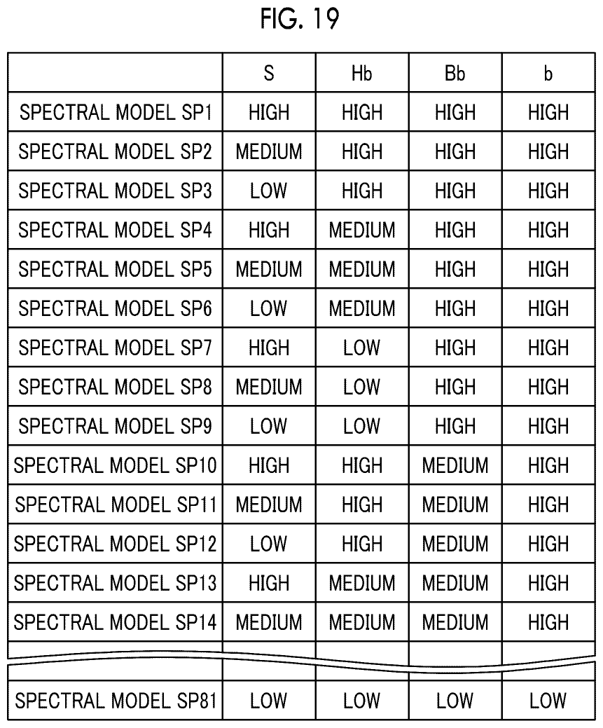

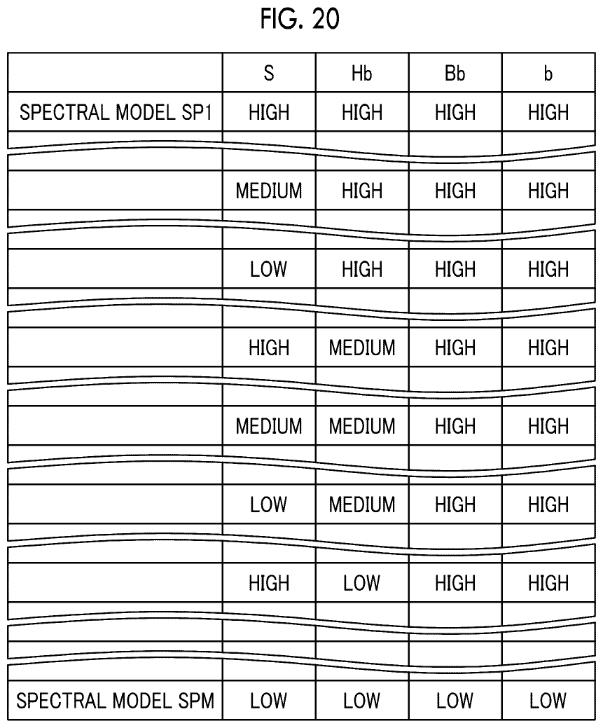

Specifically, 16 spectral models are used as spectral models that are minimally required for the calculation of the biological parameters (M=16). In this case, as shown in FIG. 18, a total of 16 spectral models SP1 to SP16 are used for two cases of the oxygen saturation, “high oxygen saturation” and “low oxygen saturation”, two cases of the hemoglobin concentration, “high hemoglobin concentration” and “low hemoglobin concentration”, two cases of the bilirubin concentration, “high bilirubin concentration” and “low bilirubin concentration”, and two cases of the scattering wavelength-dependent parameter, “high scattering wavelength-dependent parameter” and “low scattering wavelength-dependent parameter”. In this case, 16 sets of standard spectral signals are set. In FIG. 18, “S” represents the oxygen saturation, “Hb” represents the hemoglobin concentration, “Bb” represents the bilirubin concentration, and “b” represents the scattering wavelength-dependent parameter, and “High” represents a high concentration or a high parameter, and “Low” represents a low concentration or a low parameter (the same applies to FIGS. 19 and 20 (in the case of FIGS. 19 and 20, “Medium” represents a medium concentration or an intermediate parameter)).

In addition, 81 spectral models are used as spectral models necessary for improving the calculation accuracy of the biological parameters (M=81). In this case, as shown in FIG. 19, a total of 81 spectral models SP1 to SP81 are used for three cases of the oxygen saturation, “high oxygen saturation”, “medium oxygen saturation”, and “low oxygen saturation”, three cases of the hemoglobin concentration, “high hemoglobin concentration”, “medium hemoglobin concentration”, and “low hemoglobin concentration”, three cases of the bilirubin concentration, “high bilirubin concentration”, “medium bilirubin concentration”, and “low bilirubin concentration”, and three cases of the scattering wavelength-dependent parameter, “high scattering wavelength-dependent parameter”, “medium scattering wavelength-dependent parameter”, and “low scattering wavelength-dependent parameter”. In this case, 81 sets of standard spectral signals are set.

In addition, as spectral models necessary for further improving the calculation accuracy of the biological parameters, as shown in FIG. 20, for the oxygen saturation concentration, an intermediate concentration may be set between “high oxygen saturation” and “medium oxygen saturation” and an intermediate concentration may be set between “medium oxygen saturation” and “low oxygen saturation”, and the similar intermediate concentration may be set for the hemoglobin concentration, the bilirubin concentration, and the scattering wavelength-dependent parameter. In this case, a total of M spectral models SP1 to SPM are used. In this case, M sets of standard spectral signals are set.

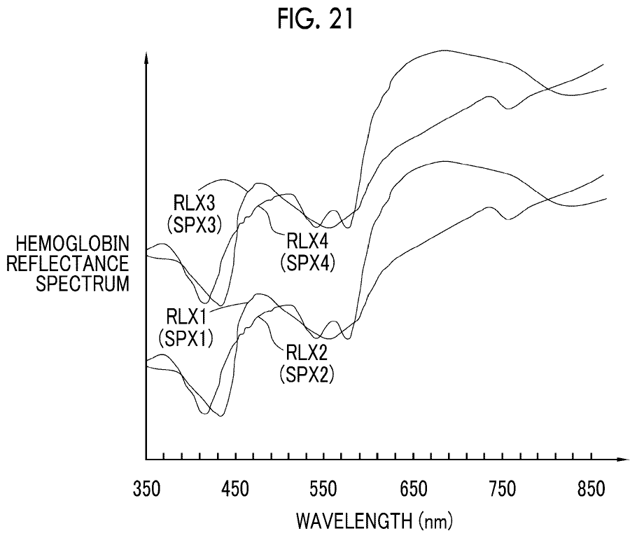

In the following, for simplification of the description, the description will be made using some spectral models SPX1 to SPX4 in the spectral models used for calculating the biological parameters. As shown in FIG. 21, a spectral reflectivity RLX1 determined by the spectral model SPX1 is determined by a biological parameter LPX1. The biological parameter LPX1 includes oxygen saturation SX1 of low oxygen saturation, a hemoglobin concentration HbX1 of high hemoglobin concentration, a medium bilirubin concentration BbX1, and a medium scattering wavelength-dependent parameter bX1.

In addition, a spectral reflectivity RLX2 determined by the spectral model SPX2 is determined by a biological parameter LPX2. The biological parameter LPX2 includes oxygen saturation SX2 of high oxygen saturation, a hemoglobin concentration HbX2 of high hemoglobin concentration, a medium bilirubin concentration BbX1, and a medium scattering wavelength-dependent parameter bX1. In addition, a spectral reflectivity RLX3 determined by the spectral model SPX3 is determined by a biological parameter LPX3. The biological parameter LPX3 includes oxygen saturation SX3 of low oxygen saturation, a hemoglobin concentration HbX3 of low hemoglobin concentration, a medium bilirubin concentration BbX1, and a medium scattering wavelength-dependent parameter bX1. In addition, a spectral reflectivity RLX4 determined by the spectral model SPX4 is determined by a biological parameter LPX4. The biological parameter LPX4 includes oxygen saturation SX4 of high oxygen saturation, a hemoglobin concentration HbX4 of low hemoglobin concentration, a medium bilirubin concentration BbX1, and a medium scattering wavelength-dependent parameter bX1.

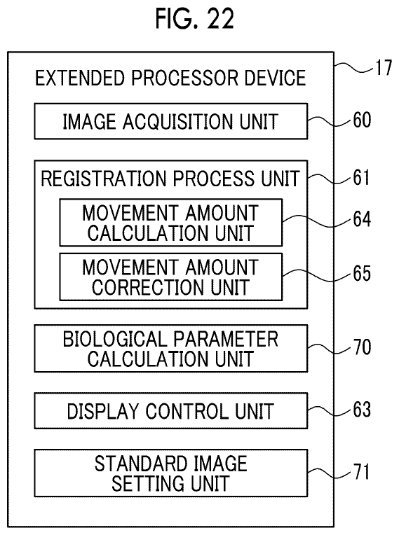

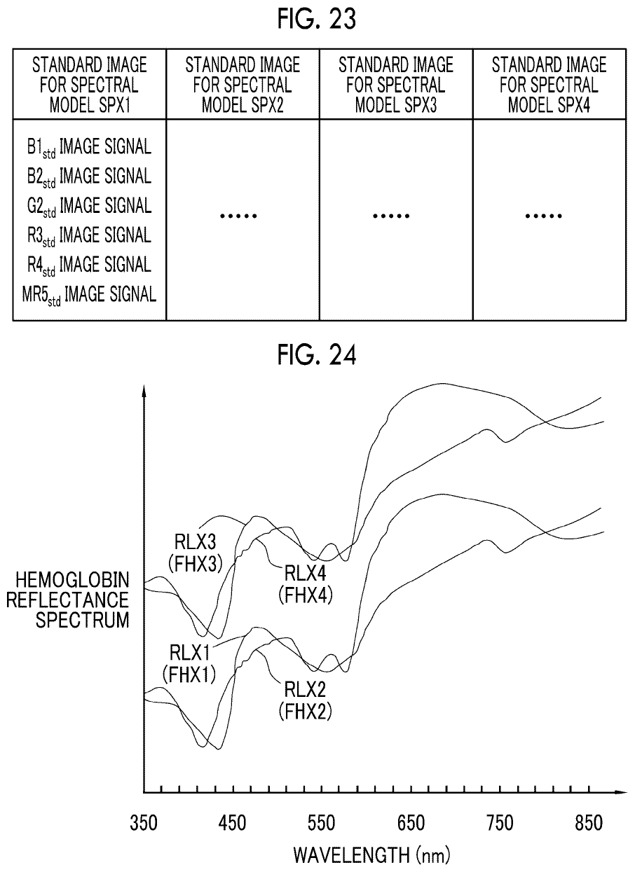

As shown in FIG. 22, a standard image setting unit 71 of the second embodiment sets a standard image for the spectral model SPX1 based on the light emission spectrum of the illumination light, the spectral reflectivity RLX1, and the spectral sensitivity of the imaging sensor 36. The method of setting the standard image is the same as that of the first embodiment. As shown in FIG. 23, the standard image for the spectral model SPX1 obtained as described above includes six image signals, that is, a B1_{std} image signal, a B2_{std} image signal, a G2_{std} image signal, an R3_{std} image signal, an R4_{std} image signal, and an MR5_{std} image signal. The standard images for the spectral models SP2, SP3, and SP4 also include six image signals, that is, a B1_{std} image signal, a B2_{std} image signal, a G2_{std} image signal, an R3_{std} image signal, an R4_{std} image signal, and an MR5_{std} image signal. Similarly, the standard images for the spectral models SPX2, SPX3, and SPX4 are set.

A biological parameter calculation unit 70 of the second embodiment selects a specific standard image having a minimum error calculation value based on an error with the aligned monochromic image among M sets of standard images. Then, a specific biological parameter determined by a spectral model corresponding to the specific standard image is calculated as the biological parameter. Specifically, in a case where four sets of standard images for the spectral models SPX1, SPX2, SPX3, and SPX4 are used, in a case in which the standard image having a minimum error calculation value based on the error with the aligned monochromic image is the standard image for the spectral model SPX1, the standard image for the spectral model SPX1 is selected as the specific standard image. In this case, the biological parameter LPX1 determined by the spectral model SPX1 is calculated as the biological parameter. That is, oxygen saturation S1 and a hemoglobin concentration Hb1 are calculated as the biological parameters. It is preferable that the error calculation value is a value obtained by using the squared error as in the first embodiment.

### Third Embodiment

In a third embodiment, in a case of calculating the biological parameters, a plurality of phantoms simulating the spectral model are used instead of the spectral model, and a biological parameter satisfying a condition is calculated as the biological parameter from among the biological parameters determined by the phantoms. In the third embodiment, M sets of standard images used for calculating the biological parameters are set for each phantom based on the light emission spectrum of the illumination light, the spectral reflectivity of the living body determined by the spectral measurement of the phantom, and the spectral sensitivity of the imaging sensor 36. The procedure other than the setting of the standard image is the same as that of the first embodiment.

Specifically, it is preferable to use phantoms corresponding to 16 spectral models shown in FIG. 18 for the phantoms that are minimally required for the calculation of the biological parameters. In this case, 16 sets of standard spectral signals are set. In addition, it is preferable to use phantoms corresponding to 81 spectral models shown in FIG. 19 for the phantoms necessary for improving the calculation accuracy of the biological parameters. In this case, 81 sets of standard spectral signals are set. In addition, it is preferable to use phantoms corresponding to M spectral models shown in FIG. 20 for the phantoms necessary for further improving the calculation accuracy of the biological parameters. In this case, M sets of standard spectral signals are set.

In the following, for simplification of the description, the description will be made using some phantoms FHX1 to FHX4 in the phantoms used for calculating the biological parameters. As shown in FIG. 24, the phantom FHX1 has a spectral reflectivity RLX1 determined by the biological parameter LPX1. The biological parameter LPX1 includes oxygen saturation SX1 of low oxygen saturation, a hemoglobin concentration HbX1 of high hemoglobin concentration, a medium bilirubin concentration BbX1, and a medium scattering wavelength-dependent parameter bX1.

In addition, the phantom FHX2 has a spectral reflectivity RLX2 determined by the biological parameter LPX2. The biological parameter LPX2 includes oxygen saturation SX2 of high oxygen saturation, a hemoglobin concentration HbX2 of high hemoglobin concentration, a medium bilirubin concentration BbX1, and a medium scattering wavelength-dependent parameter bX1. In addition, the phantom FHX3 has a spectral reflectivity RLX3 determined by the biological parameter LPX3. The biological parameter LPX3 includes oxygen saturation SX3 of low oxygen saturation, a hemoglobin concentration HbX3 of low hemoglobin concentration, a medium bilirubin concentration BbX1, and a medium scattering wavelength-dependent parameter bX1. In addition, the phantom FHX4 has a spectral reflectivity RLX4 determined by the biological parameter LPX4. The biological parameter LPX4 includes oxygen saturation SX4 of high oxygen saturation, a hemoglobin concentration HbX4 of low hemoglobin concentration, a medium bilirubin concentration BbX1, and a medium scattering wavelength-dependent parameter bX1. The above-described spectral reflectivities RLX1 to RLX4 are calculated in advance by measuring the phantoms with a spectroscopic measurement device. The calculated spectral reflectivities RLX1 to RLX4 are stored in the extended processor device 17 in advance.

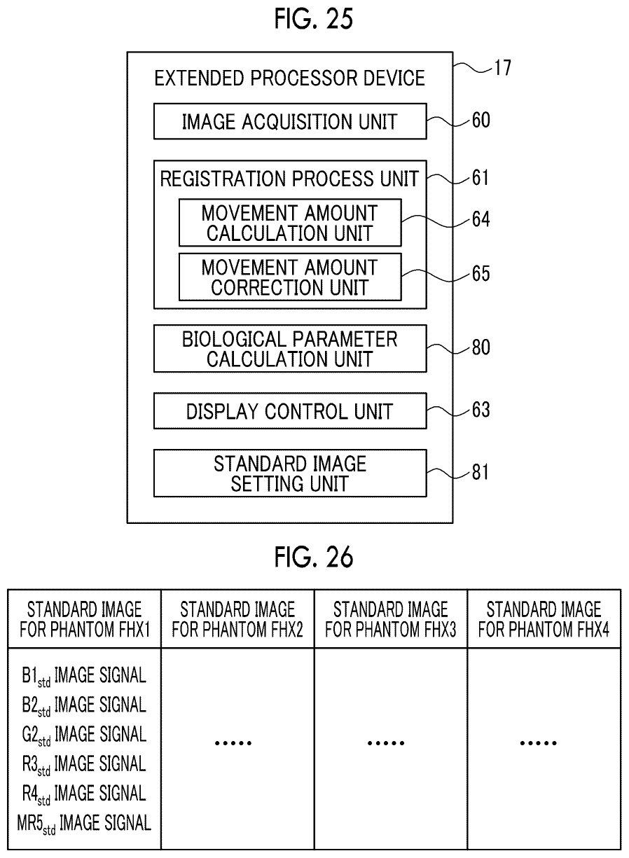

As shown in FIG. 25, a standard image setting unit 81 of the third embodiment sets a standard image for the phantom FHX1 based on the light emission spectrum of the illumination light, the spectral reflectivity RLX1, and the spectral sensitivity of the imaging sensor 36. The method of setting the standard image is the same as that of the first embodiment. As shown in FIG. 26, the standard image for the phantom FHX1 obtained as described above includes six image signals, that is, a B1_{std} image signal, a B2_{std} image signal, a G2_{std} image signal, an R3_{std} image signal, an R4_{std} image signal, and an MR5_{std} image signal. The standard images for the phantoms FHX2 to FHX4 also include six image signals, that is, a B1_{std} image signal, a B2_{std} image signal, a G2_{std} image signal, an R3_{std} image signal, an R4_{std} image signal, and an MR5_{std} image signal. Similarly, the standard images for the phantoms FHX2, FHX3, and FHX4 are set.

A biological parameter calculation unit 80 of the third embodiment selects a specific standard image having a minimum error calculation value based on an error with the aligned monochromic image among M sets of standard images. Then, a specific biological parameter determined by a spectral model corresponding to the specific standard image is calculated as the biological parameter. Specifically, in a case where four sets of standard images for the phantoms FHX1 to FHX4 are used, in a case in which the standard image having a minimum error calculation value based on the error with the aligned monochromic image is the standard image for the phantom FHX1, the standard image for the phantom FHX1 is selected as the specific standard image. In this case, the biological parameter LPX1 determined by the phantom FHX1 is calculated as the biological parameter. That is, oxygen saturation S1 and a hemoglobin concentration Hb1 are calculated as the biological parameters. It is preferable that the error calculation value is a value obtained by using the squared error as in the first embodiment.

### Fourth Embodiment

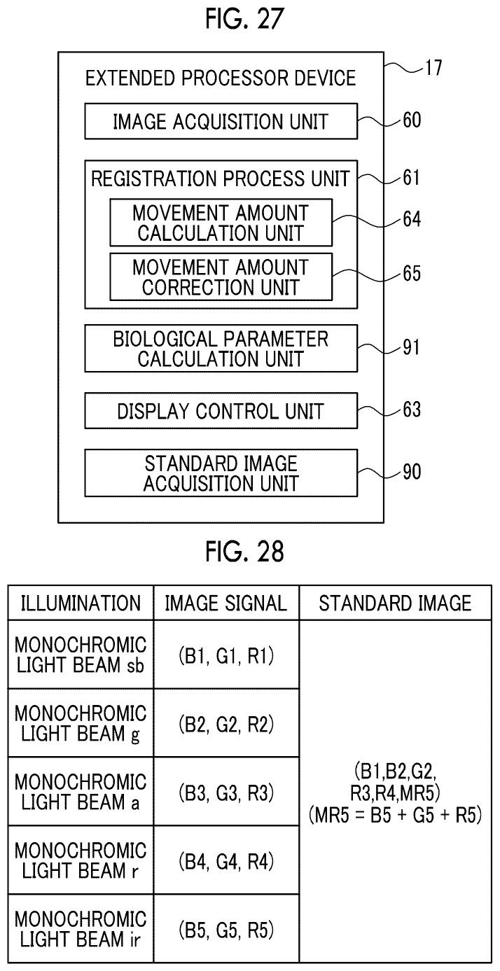

In a fourth embodiment, in a case of calculating the biological parameters, a plurality of phantoms simulating the spectral model are used instead of the spectral model, and each phantom is illuminated with a monochromic light beam and imaged to obtain a standard image. Then, a biological parameter determined by a phantom corresponding to a standard image satisfying a condition among the standard images is calculated as the biological parameter. Therefore, in the fourth embodiment, a standard image acquisition unit 90 shown in FIG. 27 acquires M sets of standard images in advance for each phantom by illuminating the phantom with a monochromic light beam and imaging the phantom. The procedure other than the acquisition of the standard image is the same as that of the first embodiment.

Specifically, it is preferable to use phantoms corresponding to 16 spectral models shown in FIG. 18 for the phantoms that are minimally required for the calculation of the biological parameters. In this case, 16 sets of standard spectral signals are acquired. In addition, it is preferable to use 81 phantoms corresponding to the spectral models shown in FIG. 19 for the phantoms necessary for improving the calculation accuracy of the biological parameters. In this case, 81 sets of standard spectral signals are acquired. In addition, it is preferable to use phantoms corresponding to M spectral models shown in FIG. 20 for the phantoms necessary for further improving the calculation accuracy of the biological parameters. In this case, M sets of standard spectral signals are acquired.

In the following, for simplification of the description, the description will be made using some phantoms FHX1 to FHX4 in the phantoms used for calculating the biological parameters. As shown in FIG. 28, the phantom FHX1 is illuminated with the monochromic light beams sb, g, a, r, and ir, and an image is captured by the imaging sensor 36 for each illumination of the monochromic light beam. By this imaging, B1, G1, and R1 image signals, B2, G2, and R2 image signals, B3, G3, and R3 image signals, B4, G4, and R4 image signals, and B5, G5, and R5 image signals are obtained. Among these image signals, the B1 image signal, the B2 image signal, the G2 image signal, the R3 image signal, the R4 image signal, and the MR5 image signal obtained by combining the B5, G5, and R5 image signals are each used as the standard image.

That is, as shown in FIG. 26, in the case of the phantom FHX1, the B1_{std} image signal, the B2_{std} image signal, the G2_{std} image signal, the R3_{std} image signal, the R4_{std} image signal, and the MR5_{std} image signal are included as the standard image. Similarly, for the phantoms FHX2 to FHX4, as in the case of the phantom FHX1, the standard image is obtained for each phantom by illuminating the phantoms FHX2 to FHX4 with the monochromic light beam and imaging the phantoms FHX2 to FHX4.

A biological parameter calculation unit 91 of the fourth embodiment selects a specific standard image having a minimum error calculation value based on an error with the aligned monochromic image among M sets of standard images. Then, a specific biological parameter determined by a phantom corresponding to the specific standard image is calculated as the biological parameter. Specifically, in a case where four sets of standard images for the phantoms FHX1 to FHX4 are used, in a case in which the standard image having a minimum error calculation value based on the error with the aligned monochromic image is the standard image for the phantom FHX1, the standard image for the phantom FHX1 is selected as the specific standard image. In this case, the biological parameter LPX1 determined by the phantom FHX1 is calculated as the biological parameter. That is, oxygen saturation S1 and a hemoglobin concentration Hb1 are calculated as the biological parameters. It is preferable that the error calculation value is a value obtained by using the squared error as in the first embodiment.

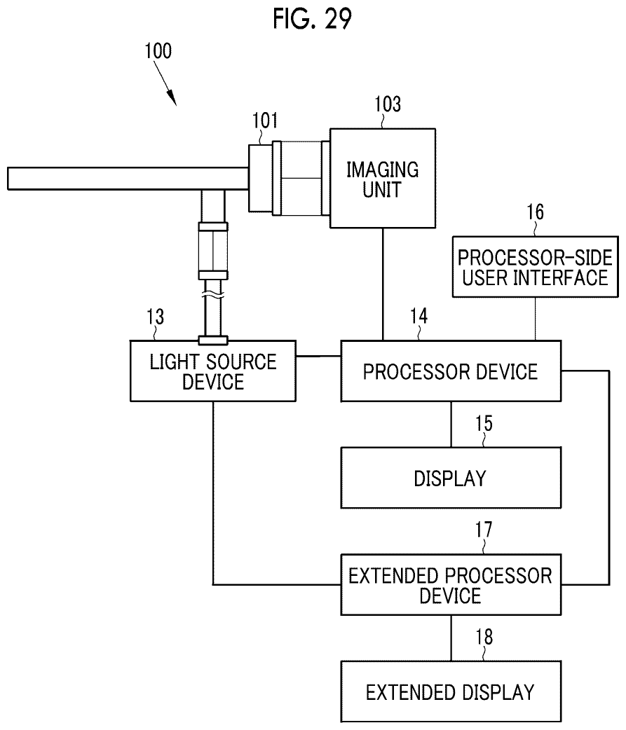

As the endoscope, in addition to the endoscope 12 which is a flexible endoscope for a digestive tract, an endoscope which is a rigid endoscope for laparoscopy may be used. In a case in which an endoscope which is a rigid endoscope is used, an endoscope system 100 shown in FIG. 29 is used. The endoscope system 100 comprises an endoscope 101, a light source device 13, a processor device 14, a display 15, a processor-side user interface 16, an extended processor device 17, and an extended display 18. In the following, in the endoscope system 100, the common parts with the endoscope system 10 will be omitted, and only the different parts will be described.

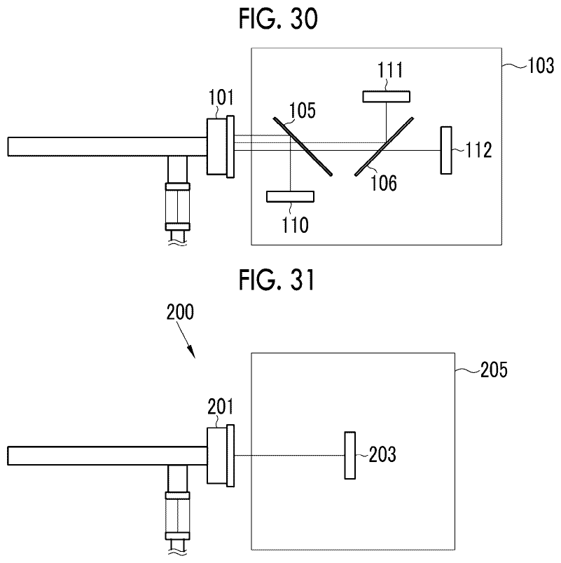

As shown in FIG. 30, an imaging unit 103 spectrally separates light from the endoscope 101 into light in a plurality of wavelength ranges and acquires an image signal based on the plurality of spectral wavelength ranges. The imaging unit 103 comprises dichroic mirrors 105, 106, and 107, and monochromic imaging sensors 110, 111, 112, and 113. The dichroic mirror 105 reflects light in a blue band among light reflected from the endoscope 101 and transmits light having a longer wavelength than the light in the blue band. The light in the blue band reflected by the dichroic mirror 105 is incident on the imaging sensor 110. The monochromic light beam sb, the light in the blue band of the monochromic light beam g, and the monochromic light beam ir are incident on the imaging sensor 110.

The dichroic mirror 106 reflects light in a green band among the light transmitted through the dichroic mirror 105 and transmits light having a longer wavelength than the light in the green band. The light in the green band reflected by the dichroic mirror 106 is incident on the imaging sensor 111. The light in the green band of the monochromic light beam g and the monochromic light beam ir are incident on the imaging sensor 111. On the other hand, the light transmitted through the dichroic mirror 106 is incident on the imaging sensor 112. The monochromic light beam r and the monochromic light beam ir are incident on the imaging sensor 112.

In the normal observation mode, the imaging sensors 110, 111, and 112 output the Bc image signal, the Gc image signal, and the Rc image signal in response to the incidence of the monochromic light beam b, the monochromic light beam g, and the monochromic light beam r. In the oxygen saturation mode, in odd-numbered frames (frames 1, 3, 5, 7, and 9), the imaging sensors 110, 111, and 112 output the Bc image signal, the Gc image signal, and the Rc image signal in response to the incidence of the monochromic light beam b, the monochromic light beam g, and the monochromic light beam r. On the other hand, in even-numbered frames of the oxygen saturation mode, in the frame 2, the imaging sensor 110 outputs the B1 image signal in response to the incidence of the monochromic light beam sb. In the frame 4, the imaging sensor 110 outputs the B2 image signal in response to the incidence of light in the blue range of the monochromic light beam g, and the imaging sensor 111 outputs the G2 image signal in response to the incidence of the monochromic light beam g.

In the frame 6, the imaging sensor 112 outputs the R3 image signal in response to the incidence of the monochromic light beam a. In the frame 8, the imaging sensor 112 outputs the R4 image signal in response to the incidence of the monochromic light beam r. In the frame 10, the imaging sensor 110 outputs the B5 image signal in response to the incidence of the monochromic light beam ir, the imaging sensor 111 outputs the G5 image signal in response to the incidence of the monochromic light beam ir, and the imaging sensor 112 outputs the R5 image signal in response to the incidence of the monochromic light beam ir. These three image signals, that is, the B5 image signal, the G5 image signal, and the R5 image signal are synthesized to generate an MR5 image signal.

In a case in which an endoscope which is a rigid endoscope for laparoscopy is used, instead of the endoscope system 100 that images the observation target using the three monochromic imaging sensors 110 to 112, an endoscope system 200 that images the observation target using another imaging method may be used. As shown in FIG. 31, in the endoscope system 200, a one-sensor type endoscope 201 for laparoscopy having one color imaging sensor 203 is used. The imaging sensor 203 is provided in an imaging unit 205 of the endoscope 201. Spectral sensitivity of the imaging sensor 203 is the same as that of the imaging sensor 36. The other points are the same as those of the endoscope system 100.

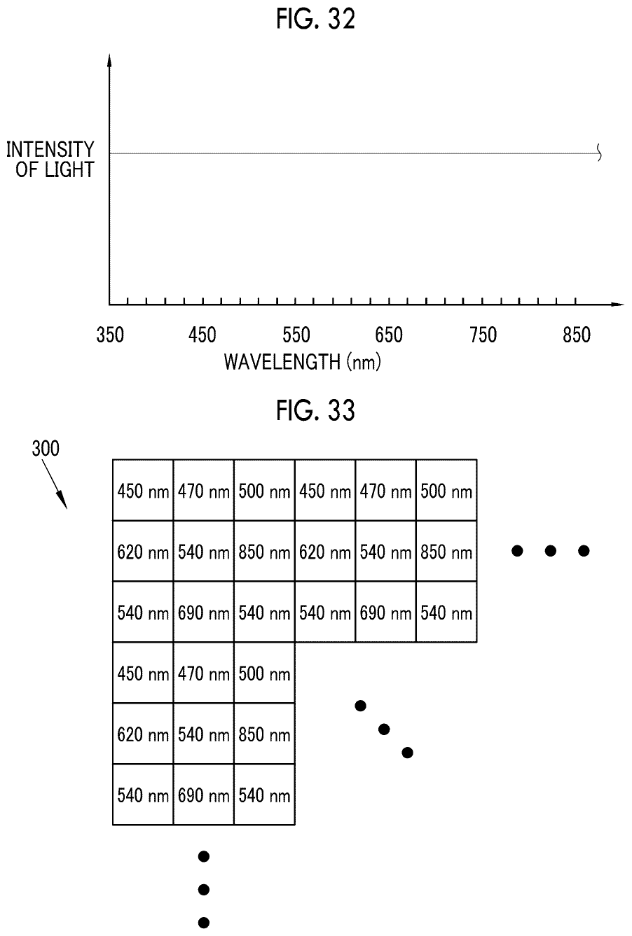

In the above-described embodiment, the aligned monochromic image is acquired by sequentially switching the monochromic light beam and performing imaging with the imaging sensor 36. Alternatively, the aligned monochromic image may be acquired by performing illumination with illumination light having a wide band and performing imaging with a spectroscopic imaging sensor that spectrally separates the illumination light having a wide band into the monochromic light beam. Specifically, as shown in FIG. 32, illumination light having a wavelength range of 300 nm to 1000 nm is used as the illumination light having a wide band. As the spectroscopic imaging sensor, a snapshot mosaic-type hyperspectral image sensor is preferably used.

In this case, as shown in FIG. 33, a spectroscopic imaging sensor 300 has a tile-like array of color filters in units of 9 pixels of 3 vertical pixels×3 horizontal pixels. In the color filter array, pixels are arranged in which color filters having central wavelengths of transmission bands of 450 nm, 470 nm, 500 nm, 540 nm, 620 nm, 690 nm, and 850 nm are provided. In FIG. 33, “450 nm, 470 nm, 500 nm, 540 nm, 620 nm, 690 nm, and 850 nm” represent pixels in which color filters having central wavelengths of transmission bands are provided, respectively. By imaging the observation target illuminated with the illumination light having a wide band by means of the spectroscopic imaging sensor 300, in the spectroscopic imaging sensor 300, a b image signal is output from a pixel of 450 nm, an sb image signal is output from a pixel of 470 nm, an sg image signal is output from a pixel of 500 nm, an lg image signal is output from a pixel of 540 nm, and an a image signal is output from a pixel of 620 nm, an r image signal is output from a pixel of 60 nm, and an ir image signal is output from a pixel of 850 nm.

In a state where the image signals are output from the spectroscopic imaging sensor 300 as described above, in each pixel, only the image signal of the pixel is present, and the image signal output from the other pixels is not present, resulting in a discrete image signal distribution. In response to this, demosaicing, which is interpolation processing between pixels, is performed to compensate pixel signal of other pixels for each pixel. As a result, image signals (b image signal, sb image signal, sg image signal, sl image signal, a image signal, r image signal, and ir image signal) for all wavelength ranges are present in each pixel. It is preferable to use, for example, a method disclosed in JP2023-529189A as the demosaicing.

For the image signal after the demosaicing, the b image signal is treated as a B1_{img} image signal, and the sb image signal is treated as a B2_{img} image signal. An image signal obtained by synthesizing the sg image signal and the sl image signal is treated as a G2_{img} image signal. The a image signal is treated as an R3_{img} image signal, the r image signal is treated as an R4_{img} image signal, and the ir image signal is treated as an MR5_{img} image signal. Then, the calculation of the biological parameters is performed in the same manner as in the above-described embodiment.

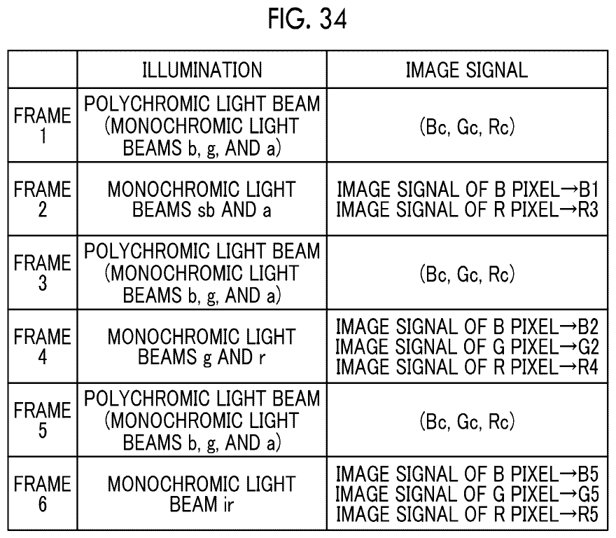

In the above-described embodiment, in the oxygen saturation mode, the monochromic light beam is emitted as the first illumination light beam between the emissions of the polychromic light beam as the second illumination light beam, but other light beam may be emitted. For example, as shown in FIG. 34, in a case in which the monochromic light beam b, the monochromic light beam g, and the monochromic light beam a are simultaneously emitted as the second illumination light beam in the frames 1, 3, and 5, the monochromic light beam sb and the monochromic light beam a may be simultaneously emitted as the first illumination light beam in the frame 2, and the monochromic light beam g and the monochromic light beam r may be simultaneously emitted as the first illumination light beam in the frame 4. Only the monochromic light beam ir is emitted as the first illumination light beam in the frame 6.

In this case, in the frame 2, the image signal output from the B pixel of the imaging sensor 36 is referred to as a B1_{img} image signal, and the image signal output from the R pixel is referred to as an R3_{img} image signal. In addition, in the frame 4, the image signal output from the B pixel of the imaging sensor is referred to as a B2_{img} image signal, the image signal output from the G pixel is referred to as a G2_{img} image signal, and the image signal output from the R pixel is referred to as an R2_{img} image signal. For the frame 6, as in the frame 10 of the above-described embodiment, the image signals output from the B pixel, the G pixel, and the R pixel of the imaging sensor 36 are referred to as a B5_{img} image signal, a G5_{img} image signal, and an R5_{img} image signal, respectively. As described above, in a case in which the first illumination light beam is emitted, the number of frames can be suppressed to six frames, that is, the frames 1 to 6.

In the above-described embodiment, hardware structures of processing units that execute various types of processing, such as the image acquisition unit 60, the registration process unit 61, the movement amount calculation unit 64, the movement amount correction unit 65, the biological parameter calculation unit 62, the display control unit 63, the standard image setting unit 66, the biological parameter calculation unit 70, the standard image setting unit 71, the biological parameter calculation unit 80, the standard image setting unit 81, the standard image acquisition unit 90, and the biological parameter calculation unit 91, are various processors as described below. The various processors include a central processing unit (CPU) that is a general-purpose processor that executes software (programs) to function as various processing units, a graphical processing unit (GPU), a programmable logic device (PLD) that is a processor capable of changing a circuit configuration after manufacture, such as a field programmable gate array (FPGA), and an exclusive electric circuit that is a processor having a circuit configuration exclusively designed to execute various kinds of processing.

One processing unit may be configured of one of these various processors, or may be configured of a combination of two or more processors of the same type or different types (for example, a plurality of FPGAs, a combination of a CPU and an FPGA, or a combination of a CPU and a GPU). In addition, a plurality of processing units may be configured of one processor. As an example in which the plurality of processing units are configured of one processor, in a polychromic manner, as typified by computers such as a client or a server, one processor is configured of a combination of one or more CPUs and software, and this processor functions as the plurality of processing units. In a monochromic manner, as represented by a system on chip (SoC), a processor that realizes the functions of the entire system including the plurality of processing units by using one integrated circuit (IC) chip is used. As described above, the various processing units are configured using one or more of the various processors as a hardware structure.

Further, the hardware structure of these various processors is more specifically an electric circuit (circuitry) in a form in which circuit elements such as semiconductor elements are combined. In addition, the hardware structure of the storage unit is a storage device such as a hard disc drive (HDD) or a solid state drive (SSD).

### EXPLANATION OF REFERENCES

10: endoscope system
12: endoscope
12a: insertion part
12b: operating part
12c: bendable part
12d: distal end part
12e: angle knob
12f: mode selector switch
12h: still image acquisition instruction switch
12i: zoom operating part
12j: forceps port
13: light source device
14: processor device
15: display
16: processor-side user interface
17: extended processor device
18: extended display
19: scope-side user interface
20: light source unit
21: light source processor
20a: b-LED
20b: sb-LED
20c: g-LED
20d: a-LED
20e: r-LED
20f: ir-LED
20g: v-LED
23: optical path combining unit
25: light guide
30: illumination optical system
31: imaging optical system
32: illumination lens
35: objective lens
36: imaging sensor
37: imaging processor
40: CDS/AGC circuit
41: A/D converter
45: DSP
50: image processing unit
51: image communication unit
52: display control unit
53: central control unit
54a: normal observation mode
54b: oxygen saturation mode
55a, 55b: curve
60: image acquisition unit
61: registration process unit
62: biological parameter calculation unit
63: display control unit
64: movement amount calculation unit
65: movement amount correction unit
66: standard image setting unit
70: biological parameter calculation unit
71: standard image setting unit
80: biological parameter calculation unit
81: standard image setting unit
90: standard image acquisition unit
91: biological parameter calculation unit
100: endoscope system
101: endoscope
103: imaging unit
105, 106: dichroic mirror
110, 111, 112: imaging sensor
200: endoscope system
201: endoscope
203: imaging sensor
205: imaging unit
300: spectroscopic imaging sensor
FHX1: phantom
FHX2: phantom
FHX3: phantom
FHX4: phantom
NP: white light image
NP1 to NP6: white light image
MP1: first monochromic image
MP2: second monochromic image
MP3: third monochromic image
MP4: fourth monochromic image
MP5: fifth monochromic image
AMP1: first aligned monochromic image
AMP2: second aligned monochromic image
AMP3: third aligned monochromic image
AMP4: fourth aligned monochromic image
AMP5: fifth aligned monochromic image
SPX1: spectral model
SPX2: spectral model
SPX3: spectral model
SPX4: spectral model
OP: oxygen saturation image
RLX1: spectral reflectivity
RLX2: spectral reflectivity
RLX3: spectral reflectivity
RLX4: spectral reflectivity
GOP: internal digestive tract oxygen saturation image
SOP: serous membrane-side oxygen saturation image
BF: B color filter
GF: G color filter
RF: R color filter
Bc: Bc image signal
Gc: Gc image signal
Rc: Rc image signal
ST010 to ST100: step

What is claimed is:

1. An endoscope system comprising:
a light source device that alternately emits one of a plurality of first illumination light beams and a second illumination light beam having a wider band than the first illumination light beams as illumination light;
an endoscope including an imaging sensor that images an observation target illuminated with the illumination light;
a processor device including a processor that is configured to generate a biological parameter image based on an endoscope image obtained by the imaging sensor and perform control of displaying the biological parameter image on a display; and
the display,
wherein the plurality of first illumination light beams are light beams in wavelength ranges having different dependences on oxygen saturation, and
the processor is configured to
generate an aligned first image by performing a registration process on a first endoscope image obtained by imaging the observation target illuminated with the first illumination light beam, based on a plurality of second endoscope images obtained by imaging the observation target illuminated with the second illumination light beam,
acquire an aligned first image set including a plurality of the aligned first images,
calculate oxygen saturation of the observation target based on the acquired aligned first image set, and
generate the biological parameter image based on the oxygen saturation.

2. The endoscope system according to claim 1,
wherein the registration process includes a movement amount calculation process and a movement amount correction process,
the movement amount calculation process is a process of calculating a movement amount of the first endoscope image based on the plurality of second endoscope images, and
the movement amount correction process is a process of generating the aligned first image by performing correction based on the movement amount on the first endoscope image of which the movement amount is calculated.

3. The endoscope system according to claim 1,
wherein the light source device alternately emits the second illumination light beam and one of the plurality of first illumination light beams for each frame.

4. The endoscope system according to claim 1,
wherein a central wavelength of each of the plurality of first illumination light beams is any one of 450 nm, 470 nm, 540 nm, 620 nm, 690 nm, or 850 nm.

5. The endoscope system according to claim 1,
wherein there are five first illumination light beams, and
central wavelengths of the first light beams are 470 nm, 540 nm, 620 nm, 690 nm, and 850 nm.

6. The endoscope system according to claim 1,
wherein the light source device illuminates the observation target with light-emitting units that emit all of the plurality of first illumination light beams in a preset order, and
the processor is configured to generate the aligned first image set based on a plurality of the first endoscope images obtained in the light-emitting units.

7. The endoscope system according to claim 6,
wherein the light source device illuminates the observation target with the light-emitting units that emit all of the plurality of first illumination light beams in ascending order of a central wavelength.

8. The endoscope system according to claim 1,
wherein the second illumination light beam is generated by emitting two or more first illumination light beams among the plurality of first illumination light beams.

9. The endoscope system according to claim 1,
wherein the second illumination light beam is generated by simultaneously emitting first illumination light beams having central wavelengths of 450 nm, 540 nm, and 620 nm among the plurality of first illumination light beams.

10. The endoscope system according to claim 2,
wherein the movement amount calculation process is a process of calculating the movement amount of the first endoscope image based on two second endoscope images obtained in frames immediately before and after the first endoscope image.

11. The endoscope system according to claim 1,
wherein the processor is configured to calculate the oxygen saturation of the observation target for each pixel of the aligned first image included in the aligned first image set.

12. The endoscope system according to claim 1,
wherein the endoscope system has a normal observation mode in which the light source device continuously emits the second illumination light beam and the processor performs control of continuously displaying the second endoscope image on the display, and a biological parameter image acquisition mode in which the light source device alternately emits the second illumination light and one of the plurality of first illumination light beams and the processor performs control of displaying the biological parameter image on the display, and
the endoscope includes a switching operation unit for switching between the normal observation mode and the biological parameter image acquisition mode, the switching operation unit being operated by an endoscope operator.

13. The endoscope system according to claim 12,
wherein, the processor is configured, in a case in which the endoscope operator switches a mode from the normal observation mode to the biological parameter image acquisition mode by operating the switching operation unit, to generate the biological parameter image based on the aligned first image set acquired first after the switching, perform control of displaying the generated biological parameter image on the display, and then automatically return to the normal observation mode.

14. The endoscope system according to claim 2,
wherein the light source device illuminates the observation target with light-emitting units that emit all of the plurality of first illumination light beams in a preset order, and
the processor is configured to
calculate a total movement amount obtained by adding up movement amounts in a plurality of the first endoscope images obtained in the light-emitting units,
acquire the aligned first image set including the plurality of aligned first images obtained in the light-emitting units in a case in which the total movement amount is within a preset range, and
calculate the oxygen saturation based on the acquired aligned first image set.

15. The endoscope system according to claim 2,
wherein the light source device illuminates the observation target with light-emitting units that emit all of the plurality of first illumination light beams in a preset order, and
the processor is configured to
calculate a total movement amount obtained by adding up movement amounts in a plurality of the first endoscope images obtained in the light-emitting units, and
perform adjustment to reduce the resolution of the aligned first image based on which the oxygen saturation of the observation target is calculated such that the degree of resolution reduction increases as the total movement amount increases.

16. A method of generating a biological parameter image executed in an endoscope system including a light source device, an endoscope, and a processor device, in which the light source device alternately emits one of a plurality of first illumination light beams and a second illumination light beam having a wider band than the first illumination light beams as illumination light, the endoscope includes an imaging sensor that images an observation target illuminated by the light source device, the processor device includes a processor that is configured to generate a biological parameter image based on an endoscope image obtained by the imaging sensor, and the plurality of first illumination light beams are light beams in wavelength ranges having different dependences on oxygen saturation, the method comprising:
via the processor,
a step of generating an aligned first image by performing a registration process on a first endoscope image obtained by imaging the observation target illuminated with the first illumination light beam, based on a plurality of second endoscope images obtained by imaging the observation target illuminated with the second illumination light beam;
a step of acquiring an aligned first image set including a plurality of the aligned first images;
a step of calculating oxygen saturation of the observation target based on the aligned first image set; and
a step of generating the biological parameter image based on the oxygen saturation.

17. A non-transitory computer readable medium for storing a computer-executable program for an endoscope system including a light source device that alternately emits one of a plurality of first illumination light beams and a second illumination light beam having a wider band than the first illumination light beams as illumination light, an endoscope including an imaging sensor that images an observation target illuminated with the illumination light, and a processor device including a processor that generates a biological parameter image based on an endoscope image obtained by the imaging sensor and that performs control of displaying the biological parameter image on a display, the computer-executable program being executed by the processor and causing the processor to execute a function of:
generating an aligned first image by performing a registration process on a first endoscope image obtained by imaging the observation target illuminated with the first illumination light beam, based on a plurality of second endoscope images obtained by imaging the observation target illuminated with the second illumination light beam;
acquiring an aligned first image set including a plurality of the aligned first images;
calculating oxygen saturation of the observation target based on the aligned first image set;
generating the biological parameter image based on the oxygen saturation; and
performing control of displaying the biological parameter image on the display.
